(19) United States
(12) Patent Application Publication
(10) Pub. No.: US 2006/0211083 A1
(43) Pub. Date: Sep. 21, 2006

(54) Products and processes for in vitro synthesis of biomolecules

(76) Inventors: Federico Katzen, Carlsbad, CA (US); Wieslaw Kudlicki, Carlsbad, CA (US); Julia Fletcher, Carlsbad, CA (US)

Correspondence Address: Bruce D. Grant;BioTechnology Law Group, Suite E, 527 N. Highway 101, Solana Beach, CA, 92075-1173, US

(21) Appl. No.: 11/336,644

(22) Filed: Jan. 20, 2006

Related U.S. Application Data
(60) Provisional application No. 60/645,891, filed on Jan. 21, 2005.

(51) Int. Cl. C12P 21/06; C07K 14/47

(52) U.S. Cl. 435/68.1; 530/395

(57) ABSTRACT

Provided herein are products and processes for efficiently synthesizing biomolecules in vitro using cell-free extracts derived from mammalian cells and insect cells. In an embodiment, provided is a process for preparing a cell-free extract from insect cells and mammalian cells that efficiently synthesizes post-translationally modified target proteins (e.g., glycosylated target proteins). In some embodiments, a ribonucleic acid is synthesized in vitro that comprises a cap, a 5′ untranslated region comprising an 18S rRNA binding ribonucleotide sequence, and a target ribonucleotide sequence. It has been determined that such ribonucleic acids result in efficient in vitro synthesis of a target protein using cell-free extracts derived from non-rabbit mammalian cells and insect cells.

### RELATED PATENT APPLICATION

This patent application claims the benefit of U.S. Provisional Application No. 60/645,891 entitled “Products and Processes for in vitro Synthesis of Biomolecules,” filed Jan. 21, 2005, naming Federico Katzen and Wieslaw Antoni Kudlicki as inventors and designated by attorney docket no. INV-1001-PV, which is herein incorporated by reference in its entirety.

### FIELD OF THE INVENTION

Provided herein are products, kits and processes for synthesizing biomolecules, such as ribonucleic acids generated by in vitro transcription and proteins synthesized by in vitro translation.

### BACKGROUND

A cell synthesizes native proteins in vivo from deoxyribonucleic acid (DNA). DNA first is transcribed into a complementary ribonucleic acid (RNA) that comprises a ribonucleotide sequence encoding the protein. RNA then directs translation of the encoded protein by interaction with various cellular components, such as ribosomes. In prokaryotic cells (e.g., bacteria) transcription and translation are “coupled,” whereby RNA is translated into protein at the same time it is transcribed from the DNA. In eukaryotic cells (e.g., animals, plants) the two processes are separate: DNA is transcribed into RNA inside the cell nucleus, the RNA is processed into message RNA (mRNA) and mRNA then is transported outside the nucleus to the cytoplasm where it is translated into protein. Eukaryotic cells include enzymes that incorporate a methylated guanosine cap on the 5′ end of the mRNA. It has been shown that such caps participate in translation as removal of the cap decreases translation efficiency.

Advances in recombinant molecular biology methodology allow researchers to isolate DNA and RNA from organisms and synthesize encoded RNA and protein products. Isolated DNA sometimes is incorporated into recombinant DNA constructs for expression in intact cells, and cell-free systems have been developed for transcribing RNA and translating proteins in vitro. In the latter in vitro systems, a cell-free extract often is prepared from cells and typically is contacted with a recombinant DNA or RNA that encodes a protein of interest. Commonly utilized cell-free extracts are prepared from E. coli bacteria, wheat germ, and rabbit reticulocytes. In vitro translation can be performed in a variety of reaction systems, which include batch reaction systems where reactants generally are brought together in closed systems; continuous flow systems, where reactants are added and products are removed from a reaction chamber by continuous flow; and continuous exchange systems, where reactants can diffuse across a semi-permeable membrane.

### SUMMARY

Many candidate biomolecules for in vitro synthesis are of eukaryotic origin, often of mammalian origin, and many candidate proteins are modified post-translationally in vivo. For example, some candidate proteins are glycosylated in vivo. Cell-free in vitro synthesis systems derived from cells other than mammalian or insect cells sometimes do not include components requisite for efficient synthesis of such proteins (e.g., codons in the template nucleic acid and tRNAs in the cell-free extract sometimes are not compatible), and sometimes do not include components requisite for efficient or appropriate post-translational modification (e.g., enzymes and other modification components may not be present in the cell-free extract or may not be present in sufficient quantities). Thus, provided herein are products and processes for efficiently synthesizing biomolecules in vitro, which often utilize cell-free extracts derived from mammalian cells and insect cells. In an embodiment, provided is a process for preparing a cell-free extract from insect cells or mammalian cells that efficiently synthesizes post-translationally modified target proteins and target peptides (e.g., glycosylated target proteins). In some embodiments, a ribonucleic acid template is synthesized in vitro that comprises an exogenous cap, a 5′ untranslated region comprising a translational enhancer sequence, and a target ribonucleotide sequence. It has been determined that ribonucleic acids comprising a tobacco mosaic virus (TMV) omega sequence translational enhancer sequence and an exogenous cap result in efficient in vitro synthesis of a target protein or target peptide using cell-free extracts derived from nonplant cells, including cultured cells, mammalian cells, and insect cells.

Accordingly, provided is a method for preparing a cell-free extract from eukaryotic cells, such as insect cells or mammalian cells, which comprises rupturing the cells using pressure or shear forces and removing intact cells, whereby a cell-free extract is prepared. Also provided is a method for preparing a cell-free extract from cultured cells, consisting essentially of rupturing cultured animal cells, such as cultured insect cells, avian cells, or mammalian cells, and removing intact cells, whereby a cell-free extract that can be used for in vitro translation is prepared. In certain embodiments, the process for preparing the cell-free extract does not include a chromatography step. In such methods, the resulting cell-free extract often comprises functional components for post-translational modification, such as glycosylation. In some preferred embodiments, the cell extract is made from cultured cells. In some embodiments, the cultured cells used to make the extract are insect cells.

Featured also is a method for synthesizing a target protein or target peptide in a cell-free system, comprising: contacting a nucleic acid that encodes a target protein or target peptide with a cell-free extract, where the cell-free extract for in vitro translation is prepared by a process which comprises rupturing eukaryotic cells, such as insect cells or mammalian cells, using physical forces and removing intact cells, whereby the target protein or target peptide is synthesized. In certain embodiments, the process for preparing the cell-free extract does not include a chromatography step. Also featured is a method for synthesizing a target protein or target peptide in a cell-free system, comprising: contacting a nucleic acid that encodes a target protein or target peptide with a cell-free extract, where the cell-free extract is prepared by a process consisting essentially of animal cells and removing intact cells, whereby the target protein or target peptide is synthesized. In such processes, the protein or peptide sometimes is glycosylated.

Some embodiments are directed to a method for synthesizing a glycosylated target protein or glycosylated target peptide in a cell-free system, comprising: contacting a nucleic acid that encodes a target protein or target peptide with a cell-free extract, where the cell-free extract is prepared by a process which comprises rupturing eukaryotic cells, such as insect cells, avian cells, or mammalian cells, using physical forces to lyse the cells and removing intact cells, whereby a glycosylated target protein or target peptide is synthesized. Certain embodiments are directed to a method for synthesizing a glycosylated target protein or target peptide in a cell-free system, comprising: contacting a nucleic acid that encodes a target protein or target peptide with a cell-free extract, where the cell-free extract is prepared by a process which consists essentially of rupturing the cells and removing intact cells, whereby glycosylated target protein or target peptide is synthesized. In certain embodiments, the process for preparing the cell-free extract does not include a chromatography step. In some embodiments the eukaryotic cells from which the cell-free extract is made are cultured cells. In some preferred embodiments, the cultured cells from which the cell-free extract is made are mammalian cells, avian cells, or insect cells. In some preferred embodiments, the cultured cells from which the cell-free extract is made are insect cells.

In the foregoing methods, the cells often are ruptured by mechanical shear (e.g., using a manual homogenizer, French Press, or Emulsiflex apparatus). The cells often are ruptured at a pressure of about 150 pounds per square inch or greater, and sometimes are ruptured at a pressure of about 175 pounds per square inch or greater, about 200 pounds per square inch or greater, about 500 pounds per square inch or greater, about 1,000 pounds per square inch or greater, or about 10,000 pounds per square inch or greater. In some embodiments, the nucleic acid is a deoxyribonucleic acid and the deoxyribonucleic acid sometimes encodes a ribonucleic acid comprising a 5′ untranslated ribonucleotide sequence comprising a translational enhancer sequence and a target ribonucleotide sequence. In certain embodiments the nucleic acid is a ribonucleic acid, which sometimes comprises a 5′ untranslated ribonucleotide sequence comprising a translational enhancer sequence, and a target ribonucleotide sequence. In certain embodiments the ribonucleic acid comprises a cap, such as an exogenous cap, for example.

Also featured is a composition comprising: a cell-free extract and an isolated ribonucleic acid comprising an exogenous cap, a 5′ untranslated ribonucleotide sequence comprising a translational enhancer sequence, and a target ribonucleotide sequence, in which the cell-fee extract is not derived from plant cells. In some embodiments, the cell-free extract is derived from cultured cells. In some embodiments, the translational enhancer sequence is the TMV omega sequence (or sequences substantially homologous or related to the TMV omega sequence). Provided also is a composition, comprising: a non-plant eukaryotic cell comprising a ribonucleic acid, wherein the ribonucleic acid comprises a cap, a 5′ untranslated ribonucleotide sequence comprising the TMV omega sequence (or sequences substantially homologous or related to the TMV omega sequence), and a target ribonucleotide sequence.

Also provided are kits comprising: one or more containers, a cell-free extract derived from eukaryotic cells in which the extracts are made essentially by physical rupture of the cells and removal of intact cells, and one or more buffers, enzymes, energy sources, inhibitors, or amino acids that can be used in a translation reaction. Kits can comprise one or more containers, a cell-free extract derived from cultured eukaryotic cells in which the extracts are made essentially by physical rupture of the cells and removal of intact cells, and one or more buffers, enzymes, energy sources, inhibitors, or amino acids that can be used in a translation reaction. Kits can further include one or more deoxyribonucleic acid molecules that encodes a ribonucleic acid which comprises a 5′ untranslated region comprising a translational enhancer sequence and a target ribonucleotide sequence and/or one or more insertion elements. Nucleic acids having one or more insertion elements are useful for cloning a variety of nucleotide sequences that encode membrane proteins, often referred to herein as “open reading frames” (ORFs), and ORFs from a collection may be cloned into such nucleic acids for in vitro translation. A nucleic acid sometimes comprises a tag element. A nucleic acid also may include a stop codon between the insertion element(s) or protein-encoding nucleotide sequence(s) and the tag element, which is useful for optionally expressing a protein or peptide with or without the tag in a system comprising one or more suppressor tRNAs. In some embodiments, the kit also comprises an RNA polymerase, a nucleic acid that encodes an RNA polymerase, an enzyme that transfers a cap to the ribonucleic acid, a nucleic acid that encodes an enzyme that transfers a cap to the ribonucleic acid, a cap, one or more dialysis components, a non-plant eukaryotic cell, an agent that transfers a nucleic acid into a cell, instructions for transcribing the ribonucleic acid in vitro or in vivo, instructions for translating the target ribonucleotide sequence in vitro and/or instructions for translating the target ribonucleotide sequence in vivo. In certain embodiments, the 5′ untranslated ribonucleotide sequence is heterologous to the target ribonucleotide sequence; the 5′ untranslated ribonucleotide sequence is heterologous to the cells from which the cell extract is derived; the 5′ untranslated ribonucleotide sequence is from a virus that does not substantially infect cells from which the cell-free extract is derived; the translational enhancer sequence is heterologous to the target ribonucleotide sequence; the translational enhancer sequence is heterologous to the cells from which the cell extract is derived; and/or the translational enhancer sequence is from a virus that does not substantially infect cells from which the cell-free extract is derived. In certain embodiments, the 5′ untranslated ribonucleotide sequence is the TMV omega sequence, or sequences substantially homologous to or related to the TMV omega sequence that act as translational enhancers.

Featured also is a method for cell-free translation of a protein or peptide, comprising: contacting in a system a cell-free extract derived from non-plant eukaryotic cells with a ribonucleic acid comprising a 5′ untranslated region comprising a TMV omega sequence (or sequences substantially homologous to or related to the TMV omega sequence) and a target ribonucleotide sequence; whereby a protein or peptide is translated from the target ribonucleotide sequence. The ribonucleic acid in such methods sometimes comprises an exogenous cap. Also provided is a method for cell-free translation of a protein or peptide, comprising: contacting a cell-free extract derived from non-plant eukaryotic cells with a deoxyribonucleic acid that encodes a ribonucleic acid which comprises a 5′ untranslated region comprising the TMV omega sequence and a target ribonucleotide sequence; whereby the protein or peptide is translated from the target ribonucleotide sequence. The latter method sometimes comprises contacting the ribonucleic acid with an exogenous cap. In such methods, the protein or peptide translated from the target ribonucleotide sequence sometimes is glycosylated. The methods also sometimes further comprise contacting the cell-free system with an energy source and/or contacting the cell-free system with one or more dialysis components.

Provided also is a method for synthesizing a protein or peptide in non-plant cells, comprising: maintaining or growing non-plant cells comprising a ribonucleic acid which comprises a 5′ untranslated region comprising a TMV omega sequence and a target ribonucleotide sequence; whereby a protein or peptide encoded by the target ribonucleotide sequence is synthesized from the ribonucleic acid. The ribonucleic acid sometimes is contacted with an exogenous cap. In some embodiments, the ribonucleic acid is transfected into the cells or the ribonucleic acid is transcribed from a deoxyribonucleic acid transfected into the cells that encodes the translational enhancer sequence and the target ribonucleotide sequence. Also provided is a method for synthesizing a ribonucleic acid in cells, comprising: maintaining or growing non-plant eukaryotic cells comprising a deoxyribonucleic acid encoding a ribonucleic acid comprising a 5′ untranslated region comprising a TMV omega sequence and a target ribonucleotide sequence under conditions suitable for ribonucleic acid synthesis; and contacting the cells with an exogenous cap; whereby a ribonucleic acid is synthesized that comprises the cap, the a TMV omega sequence and the target ribonucleotide sequence.

In the preceding embodiments, a cell-free extract sometimes is derived from hamster cells, monkey cells, avian cells, human cells, or insect cells and a non-plant cell sometimes is a hamster cell, monkey cell, avian cell, human cell, insect cell, or Spodoptera cell, for example. The translational enhancer sequence sometimes is an 18S rRNA-binding sequence (e.g., a 40S ribosome subunit binding sequence), and sometimes is an internal ribosome entry site (IRES) sequence. The translational enhancer sequence sometimes is 11 or more nucleotides in length, sometimes is a viral nucleotide sequence, and sometimes is a Tobacco Mosaic Virus omega sequence. In some embodiments, the 5′ untranslated ribonucleotide sequence is heterologous to the target ribonucleotide sequence; the 5′ untranslated ribonucleotide sequence is heterologous to the cells from which the cell extract is derived; and/or the 5′ untranslated ribonucleotide sequence is from a virus that does not substantially infect cells from which the cell-free extract is derived. In certain embodiments, the translational enhancer sequence is heterologous to the target ribonucleotide sequence; the translational enhancer sequence is heterologous to the cells from which the cell extract is derived; and/or the translational enhancer sequence is from a virus that does not substantially infect cells from which the cell-free extract is derived.

The target ribonucleotide sequence sometimes is a siRNA, shRNA, interfering RNA, antisense RNA or ribozyme, and sometimes encodes a protein or peptide. In some embodiments, the protein or peptide comprises a tag, which sometimes is selected from the group consisting of glutathione S-transferase, maltose binding protein, V5 protein, a fluorescent protein, a polyhistidine sequence, and a cysteine-rich sequence. A cap sometimes is an exogenous cap, often comprises a methylated nucleotide base, and sometimes is a cap disclosed in Table 1. In some embodiments, the ribonucleic acid is isolated and added to an in vitro translation system, and alternatively, the ribonucleic acid is encoded by a deoxyribonucleic acid, often an isolated deoxyribonucleic acid, added to an in vitro transcription/translation system. In certain embodiments, the deoxyribonucleic acid includes one or more insertion elements useful for cloning an ORF (e.g., an ORF from a collection), and sometimes the deoxyribonucleic acid or ribonucleic acid includes a stop codon between a tag element and an ORF useful for expressing an ORF with or without a tag in a system comprising one or more suppressor tRNA.

These and other embodiments are described in greater detail in the description which follows.

### BRIEF DESCRIPTION OF THE DRAWINGS

FIG. 1 shows an autoradiograph of a gel of the gp120 gene and human ORFs translated in a cultured cell lysate system, in which samples treated and not treated with endoglycosidase F are run side-by-side. ORFs are listed in Table 1.

FIG. 2 shows an autoradiograph of a gel comparing processed and unprocessed forms of beta lactamase translated in the insect lysate and wheat germ translation systems.

FIG. 3 shows the amount of translation of constructs that contain the TMV omega sequence and do not contain the TMV omega sequence as measured by luciferase assay.

### DETAILED DESCRIPTION

The present invention relates to in vitro synthesis of proteins and nucleic acids. The invention includes synthesis systems, methods and kits embodying one or more of the features of the present invention. Synthesis systems of the present invention include components necessary for synthesizing target nucleic acids and/or target proteins from nucleic acid templates. In vitro synthesis systems described herein provide efficient synthesis in a cell-free environment. The methods of the present invention are useful for making systems or compositions described herein and for using the systems of the present invention to produce product molecules of interest. The kits of the present invention allow the artisan to practice of the in vitro synthesis systems described herein.

Template Nucleic Acid

A general system includes a nucleic acid template that encodes a nucleic acid of interest (e.g., RNA or mRNA) and/or protein or peptide of interest. Nucleotide sequences that encode a nucleic acid, protein or peptide of interest sometimes are referred to herein as “target” nucleotide sequences, and the protein or peptide of interest sometimes is referred to as a “target” protein or peptide. A nucleic acid template can be any template, such as DNA, cDNA, RNA or mRNA, for example, and can be in any form (e.g., linear, circular, supercoiled, single-stranded, double-stranded, and the like). A template nucleic acid sometimes is a plasmid, phage, autonomously replicating sequence (ARS), centromere, artificial chromosome or other nucleic acid able to replicate or be replicated in vitro or in a host cell. Such templates are selected for their ability to guide production of a desired protein or nucleic acid molecule. The desired protein can be any polymer of amino acids encodable by a nucleic acid template, and a protein sometimes is referred to herein as a “polypeptide.” The protein can be further processed coincident with synthesis or after synthesis. When desired, the system can be altered as known in the art such that codons will encode for a different amino acid than is normal, including unconventional or unnatural amino acids (including detectably labeled amino acids).

A template nucleic acid comprises certain elements, which often are selected according to the type of target nucleotide sequence transcribed and/or translated. A template nucleic acid includes one or more or all of the following nucleotide elements: one or more promoter elements, one or more 5′ untranslated regions (5′ UTRs), one or more regions into which a target nucleotide sequence may be inserted (an “insertion element”), one or more target nucleotide sequences, one or more 3′ untranslated regions (3′ UTRs), and a selection element. A template nucleic acid is provided with one or more of such elements and other elements may be inserted into the nucleic acid before the template is contacted with an in vitro transcription and/or translation system. In some embodiments, a provided template nucleic acid comprises a promoter, 5′ UTR, optional 3′ UTR and insertion element(s) by which a target nucleotide sequence is inserted (i.e., cloned) into the template. In certain embodiments, a provided template nucleic acid comprises a promoter, insertion element(s) and optional 3′ UTR, and a 5′ UTR/target nucleotide sequence is inserted with an optional 3′ UTR. The elements can be arranged in any order suitable for in vitro transcription and/or translation, and in some embodiments a template nucleic acid comprises the following elements in the 5′ to 3′ direction: (1) promoter element, 5′ UTR, and insertion element(s); (2) promoter element, 5′ UTR, and target nucleotide sequence; (3) promoter element, 5′ UTR, insertion element(s) and 3′ UTR; and (4) promoter element, 5′ UTR, target nucleotide sequence and 3′ UTR.

A promoter element typically is required for DNA synthesis and/or RNA synthesis. A promoter often interacts with a RNA polymerase. A polymerase is an enzyme that catalyses synthesis of nucleic acids using a preexisting nucleic acid template. When the template is a DNA template, an RNA molecule is transcribed before protein is synthesized. Enzymes having polymerase activity suitable for use in the present methods include any polymerase that is active in the chosen system with the chosen template to synthesize protein. The cell-free extract can include a suitable polymerase, such as RNA polymerase II, SP6 RNA polymerase, T3 RNA polymerase, T7 RNA polymerase, RNA polymerase III and phage derived RNA polymerases. These and other polymerases are known and nucleic acid sequences with which they interact are known. Such sequences are readily accessed by the artisan, such as by searching one or more public or private databases, for example, and the sequences are readily adapted to template nucleic acids described herein.

A polymerase sometimes is endogenous in the cell free extract and sometimes is exogenous. The term “exogenous” as used herein generally refers to a component added to a cell-free extract or in vitro expression system. An exogenous component sometimes is an isolated component, such as an isolated protein or peptide or nucleic acid. An exogenous protein or peptide component sometimes is expressed in situ in an in vitro transcription and/or translation system from a nucleic acid encoding it, sometimes from the template nucleic acid (i.e., expressed in cis) and sometimes from another nucleic acid (i.e., expressed in trans). An exogenous component sometimes is not present in a cell-free extract, sometimes is synthetic, and sometimes is from an organism species different than the cells from which a cell-free extract is prepared (e.g., a T7 polymerase sometimes is added to an in vitro system comprising a cell-free extract prepared from mammalian or insect cells).

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, bird, insect or mammal). The artisan may select appropriate elements for the 5′ UTR based upon the transcription and/or translation system being utilized. A 5′ UTR sometimes comprises one or more of the following elements known to the artisan: translational enhancer sequence, transcription initiation site, transcription factor binding site, translation regulation site, translation initiation site, translation factor binding site, ribosome binding site, replicon, enhancer element, internal ribosome entry site (IRES), and silencer element.

A 5′ UTR in the template nucleic acid often comprises a translational enhancer nucleotide sequence. As used herein, the article “a” or “an” can refer to one or more of the elements it precedes (e.g., a nucleic acid comprising “a” translational enhancer sequence may comprise one or more, two or more, or three or more translational enhancer sequences). A translational enhancer nucleotide sequence often is located between the promoter and the target nucleotide sequence in a template nucleic acid. A translational enhancer sequence can be a sequence that binds to a ribosome, sometimes is an 18S rRNA-binding ribonucleotide sequence (i.e., a 40S ribosome binding sequence), and sometimes is an internal ribosome entry sequence (IRES). Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., http address www.interscience.wiley.com, DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence from TMV having the sequence: 5′-TATTTTTACA ACAATTACCA ACAACACAAA CAACAAACAA CATTACAATT ACTATTTACA ATAACA-3′ (SEQ ID NO:1) is included in the template nucleic acid as a translational enhancer sequence. Sequences substantially homologous to SEQ ID NO:1 are included as translational enhancers useful in the constructs and methods of the present invention, in which the TMV omega sequence-homologous translational enhancers have at least 60%, at least 70%, at least 80%, at least 90%, or at least 95% sequence identity with SEQ ID NO:1 and increase translation levels of a transcript into which they are incorporated by at least 20%, and preferably by at least 50% over that of the same transcript lacking the omega-homologous sequence. Studies determining critical sequences for translational enhancement can allow one skilled in the art to design and test TMV omega-homologous sequences as translational enhancers (for example, Gallie, et al. Nucleic Acids Research 20: 4631, 1992). Translational enhancement can be measured by detecting the amount or activity of a protein translated from an RNA that includes the enhancer sequence, for example, by autoradiography of labeled translation products, or by assays such as but not limited to luciferase assays, GUS assays, CAT assays, fluorescence detection (for example of translated fluorescent proteins or lumio-labeled proteins), beta lactamase assays, etc. Such assays are well-known in the art.

Also included are sequences related to SEQ ID NO:1 that have at least one poly (CAA) region of twenty bases or more that comprises four or more (CAA) sequences. Such poly CAA regions have been found to enhance translation in plant systems (Gallie, et al. Nucleic Acids Research 20: 4631, 1992). The present invention contemplates the use of enhancers such as the TMV omega sequence and translation-enhancing sequences related to the TMV omega sequence having one or more poly (CAA) regions in non-plant translation systems, including translation systems that use extracts of mammalian, avian, or insect cells. In exemplary embodiments, a TMV omega-related enhancer sequence includes at least one poly (CAA) region of twenty-five bases or more that comprise five or more (CAA) sequences and does not include any guanosine (G) residues, and the enhancer sequence increases translation levels of a transcript in which they are incorporated by at least 20%, and preferably by at least 50%. Such TMV omega-related enhancer sequences can be at least 50%, at least 60%, at least 70%, at least 80%, at least 90%, or at least 95% homologous to the TMV omega sequence.

In some embodiments, a translational enhancer sequence comprises one or more ARC-1 or ARC-1 like sequences, such as one of the following nucleotide sequences GCCGGCGGAG, CUCAUAAGGU, GACUUUGAUU, CGGAACCCAA, AUACUCCCCC and CCUUGCGACC, or a substantially identical sequence thereof. In certain embodiments, a translational enhancer sequence comprises an IRES sequence, such as one or more of EMBL nucleotide sequences J04513, X87949, M95825, M12783, AF025841, AF013263, AF006822, M17169, M13440, M22427, D14838 and M17446, or a substantially identical nucleotide sequence thereof. An IRES sequence may be a type I IRES (e.g., from enterovirus (e.g., poliovirus), rhinovirus (e.g., human rhinovirus)), a type II IRES (e.g., from cardiovirus (e.g., encephalomyocraditis virus), aphthovirus (e.g., foot-and-mouth disease virus)), a type III IRES (e.g., from Hepatitis A virus) or other picornavirus sequence (e.g., Paulos et al. supra, and Jackson et al., RNA 1: 985-1000 (1995)).

A 5′ UTR may comprise, consist essentially of or consist of a translational enhancer sequence. Where a 5′ UTR comprises or consists essentially of a translational enhancer sequence, the translational enhancer sequence may be homologous to another nucleotide sequence in the 5′ UTR, and in some embodiments, it is exogenous to another sequence in the 5′ UTR. A translational enhancer sequence sometimes is 10 or more nucleotides in length, 11 or more nucleotides in length, and sometimes 12 or more, 13 or more, 14 or more, 15 or more, 20 or more, 25 or more, 30 or more, 40 or more, 50 or more, 60 or more, 70 or more, 80 or more, 90 or more or 100 or more nucleotides in length. A translational enhancer sequence often is located between the 3′ end of a promoter and the 5′ end of a translated nucleotide sequence. The distance between the 3′ end of the translational enhancer sequence and the 5′ end of a translated nucleotide sequence sometimes is about 5 to about 50 nucleotides, about 10 to about 20 nucleotides, or about 15 nucleotides in length in length.

The term “about” as used herein refers to a value sometimes within 10% of the underlying parameter (i.e., plus or minus 10%), a value sometimes within 5% of the underlying parameter (i.e., plus or minus 5%), a value sometimes within 2.5% of the underlying parameter (i.e., plus or minus 2.5%), or a value sometimes within 1% of the underlying parameter (i.e., plus or minus 1%), and sometimes refers to the parameter with no variation. Thus, a distance of “about 20 nucleotides in length” includes a distance of 19 or 21 nucleotides in length (i.e., within a 5% variation) or a distance of 20 nucleotides in length (i.e., no variation) in some embodiments.

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the transcription and/or translation system being utilized. A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

A “target nucleotide sequence” as used herein encodes a nucleic acid, peptide, polypeptide or protein of interest, and may be a ribonucleotide sequence or a deoxyribonucleotide sequence. The term “nucleic acid” as used herein is generic to polydeoxyribonucleotides (containing 2′-deoxy-D-ribose or modified forms thereof), to polyribonucleotides (containing D-ribose or modified forms thereof), and to any other type of polynucleotide which is an N-glycoside of a purine or pyrimidine bases, or modified purine or pyrimidine bases. A target nucleic acid sometimes is an untranslated ribonucleic acid and sometimes is a translated ribonucleic acid. An untranslated ribonucleic acid may include, but is not limited to, a small interfering ribonucleic acid (siRNA), a short hairpin ribonucleic acid (shRNA), other ribonucleic acid capable of RNA interference (RNAi), an antisense ribonucleic acid, or a ribozyme. A translatable target nucleotide sequence (e.g., a target ribonucleotide sequence) sometimes encodes a peptide, polypeptide or protein, which are sometimes referred to herein as “target peptides,” “target polypeptides” or “target proteins.” Any peptides, polypeptides or proteins may be encoded by a target nucleotide sequence and may be selected by a person of ordinary skill in the art. Representative proteins include antibodies, enzymes, serum proteins (e.g., albumin), hormones (e.g., growth hormone, erythropoietin, insulin, etc.), cytokines, etc., and include both naturally occurring and exogenously expressed polypeptides. The term “protein” as used herein refers to a molecule having a sequence of amino acids linked by peptide bonds. This term includes fusion proteins, oligopeptides, polypeptides, cyclic peptides, polypeptides and polypeptide derivatives. A protein or polypeptide sometimes is of intracellular origin (e.g., located in the nucleus, cytosol, or interstitial space of host cells in vivo) and sometimes is a cell membrane protein in vivo.

A translatable nucleotide sequence generally is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids), and sometimes is referred to herein as an “open reading frame” (ORF). A template nucleic acid sometimes comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species, such as human, insect, nematode, bovine, equine, canine, feline, rat or mouse, for example. In some embodiments, the ORF is from a non-rabbit mammal (e.g., human).

A template nucleic acid sometimes comprises a nucleotide sequence adjacent to an ORF that is translated in conjunction with the ORF and encodes an amino acid tag. The tag-encoding nucleotide sequence is located 3′ and/or 5′ of an ORF in the template nucleic acid, thereby encoding a tag at the C-terminus or N-terminus of the protein or peptide encoded by the ORF. Any tag that does not abrogate in vitro transcription and/or translation may be utilized and may be appropriately selected by the artisan. A tag sometimes specifically binds a molecule or moiety of a solid phase or a detectable label, for example, thereby having utility for isolating, purifying and/or detecting a protein or peptide encoded by the ORF. In some embodiments, a tag comprises one or more of the following elements: FLAG (e.g., DYKDDDDKG), V5 (e.g., GKPIPNPLLGLDST), c-myc (e.g., EQKLISEEDL), HSV (e.g., QPELAPEDPED), influenza hemagglutinin, HA (e.g., YPYDVPDYA), VSV-G (e.g., YTDIEMNRLGK), bacterial glutathione-S-transferase, maltose binding protein, a streptavidin- or avidin-binding tag (e.g., pcDNA™6 BioEase™ Gateway® Biotinylation System (Invitrogen)), thioredoxin, β-galactosidase, VSV-glycoprotein, a fluorescent protein (e.g., green fluorescent protein and its many color variants), a polylysine or polyarginine sequence, a polyhistidine sequence (e.g., His₆) or other sequence that chelates a metal (e.g., cobalt, zinc, nickel, copper), and/or a cysteine-rich sequence that binds to an arsenic-containing molecule. In certain embodiments, a cysteine-rich tag comprises the amino acid sequence CC-Xₙ-CC, wherein X is any amino acid and n is 1 to 3, and the cysteine-rich sequence sometimes is CCPGCC. In certain embodiments, the tag comprises a cysteine-rich element and a polyhistidine element (e.g., CCPGCC and His₆).

A tag often conveniently binds to a binding partner. For example, some tags bind to an antibody (e.g., FLAG) and sometimes specifically bind to a small molecule. For example, a polyhistidine tag specifically chelates a bivalent metal, such as copper, zinc, nickel, and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FlAsH™ ([4′,5′-bis(1,3,2-dithioarsolan-2-yl)fluorescein-(1,2-ethanedithiol)₂]) and ReAsH reagents (e.g., U.S. Pat. No. 5,932,474 to Tsien et al., entitled “Target Sequences for Synthetic Molecules;” U.S. Pat. No. 6,054,271 to Tsien et al., entitled “Methods of Using Synthetic Molecules and Target Sequences;” U.S. Pat. Nos. 6,451,569 and 6,008,378; published U.S. Patent Application 2003/0083373, and published PCT Patent Application WO 99/21013, all to Tsien et al. and all entitled “Synthetic Molecules that Specifically React with Target Sequences”, all incorporated by reference for all disclosure of arsenic-containing dyes, tetracys sequence tags, and protein detection). Such antibodies and small molecules sometimes are linked to a solid phase for convenient isolation of the target protein or target peptide, as described in greater detail hereafter.

A tag sometimes comprises a sequence that localizes a translated protein or peptide to a component in a transcription and/or translation system, which is referred to as a “signal sequence” or “localization signal sequence” herein. A signal sequence often is incorporated at the N-terminus of a target protein or target peptide, and sometimes is incorporated at the C-terminus. Examples of signal sequences are known to the artisan, are readily incorporated into a template nucleic acid, and often are selected according to the cells from which a cell-free extract is prepared. A signal sequence in some embodiments localizes a translated protein or peptide to a cell membrane. Examples of signal sequences include, but are not limited to, a nucleus targeting signal (e.g., steroid receptor sequence and N-terminal sequence of SV40 virus large T antigen); mitochondia targeting signal (e.g., amino acid sequence that forms an amphipathic helix); peroxisome targeting signal (e.g., C-terminal sequence in YFG from S.cerevisiae); and a secretion signal (e.g., N-terminal sequences from invertase, mating factor alpha, PHO5 and SUC2 in S.cerevisiae; multiple N-terminal sequences of B. subtilis proteins (e.g., Tjalsma et al., Microbiol.Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); lam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273)). All of these patents disclosing signal sequences are herein incorporated by reference.

A tag sometimes is directly adjacent to the amino acid sequence encoded by an ORF (i.e., there is no intervening sequence) and sometimes a tag is substantially adjacent to a the ORF encoded amino acid sequence (e.g., an intervening sequence is present) An intervening sequence sometimes includes a recognition site for a protease, which is useful for cleaving a tag from a target protein or peptide. In some embodiments, the intervening sequence is cleaved by Factor Xa (e.g., recognition site I(E/D)GR), thrombin (e.g., recognition site LVPRGS), enterokinase (e.g., recognition site DDDDK), TEV protease (e.g., recognition site ENLYFQG) or PreScission™ protease (e.g., recognition site LEVLFQGP), for example.

An intervening sequence sometimes is referred to herein as a “linker sequence,” and may be of any suitable length selected by the artisan. A linker sequence sometimes is about 1 to about 20 amino acids in length, and sometimes about 5 to about 10 amino acids in length. The artisan may select the linker length to substantially preserve target protein or peptide function (e.g., a tag may reduce target protein or peptide function unless separated by a linker), to enhance disassociation of a tag from a target protein or peptide when a protease cleavage site is present (e.g., cleavage may be enhanced when a linker is present), and to enhance interaction of a tag/target protein product with a solid phase. A linker can be of any suitable amino acid content, and often comprises a higher proportion of amino acids having relatively short side chains (e.g., glycine, alanine, serine and threonine).

A nucleic acid template sometimes includes a stop codon between a tag element and an insertion element or ORF, which can be useful for translating an ORF with or without the tag. Mutant tRNA molecules that recognize stop codons (described above) suppress translation termination and thereby are designated “suppressor tRNAs.” Suppressor tRNAs can result in the insertion of amino acids and continuation of translation past stop codons (e.g., U.S. Patent Application No. 60/587,583, filed Jul. 14, 2004, entitled “Production of Fusion Proteins by Cell-Free Protein Synthesis,”; Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, DC). A number of suppressor tRNAs are known, including but not limited to, supE, supP, supD, supF and supZ suppressors, which suppress the termination of translation of the amber stop codon; supB, gIT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. Mutations that enhance the efficiency of termination suppressors (i.e., increase stop codon read-through) have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Thus, a template nucleic acid comprising a stop codon located between an ORF and a tag can yield a translated ORF alone when no suppressor tRNA is present in the translation system, and can yield a translated ORF-tag fusion when a suppressor tRNA is present in the system. In some embodiments, the stop codon is located 3′ of an insertion element or ORF and 5′ of a tag, and the stop codon sometimes is an amber codon. Suppressor tRNA sometimes are within a cell-free extract (e.g., the cell-free extract is prepared from cells that produce the suppressor tRNA), sometimes are added to the cell-free extract as isolated molecules, and sometimes are added to a cell-free extract as part of another extract. A provided suppressor tRNA sometimes is loaded with one of the twenty naturally occurring amino acids or an unnatural amino acid (described herein). Suppressor tRNA can be generated in cells transfected with a nucleic acid encoding the tRNA (e.g., a replication incompetent adenovirus containing the human tRNA-Ser suppressor gene can be transfected into cells). Vectors for synthesizing suppressor tRNA and for translating ORFs with or without a tag are available to the artisan (e.g., Tag-On-Demand™ kit (Invitrogen Corporation, California); Tag-On-Demand™ Suppressor Supernatant Instruction Manual, Version B, 6 Jun. 2003, at http address www.invitrogen.com/content/sfs/manuals/tagondemand_supernatant_man.pdf; Tag-On-Demand™ Gateway® Vector Instruction Manual, Version B, 20 Jun., 2003 at http address www.invitrogen.com/content/sfs/manuals/tagondemand_vectors_man.pdf; and Capone et al., Amber, ochre and opal suppressor tRNA genes derived from a human serine tRNA gene. EMBO J. 4:213, 1985).

Any convenient cloning strategy known to the artisan may be utilized to incorporate an element, such as an ORF, into a template nucleic acid. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the template nucleic acid, such as an oligonucleotide primer hybridization site for PCR, for example, and others described hereafter.

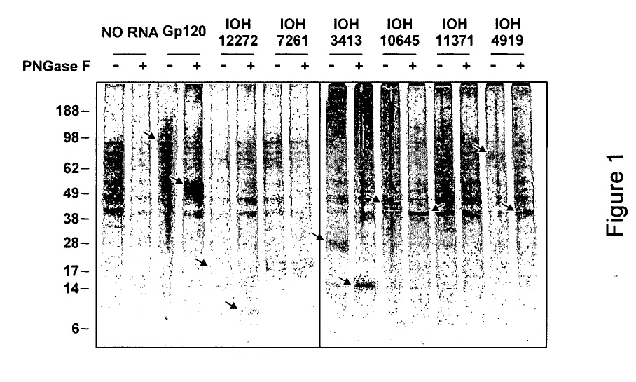

In some embodiments, the template nucleic acid includes one or more recombinase insertion sites. A recombinase insertion site is a recognition sequence on a nucleic acid molecule that participates in an integration/recombination reaction by recombination proteins. For example, the recombination site for Cre recombinase is loxp, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent appliction Ser. No. 09/517,466, filed Mar. 2, 2000, and Ser. No. 09/732,914, filed Aug. 14, 2003, and in U.S. patent publication no. 2002-0007051-A1; Landy, Curr. Opin. Biotech. 3:699-707 (1993). All references are incorporated by reference herein.). Examples of recombinase cloning nucleic acids are in Gateway® systems (Invitrogen, California), which include at least one recombination site for cloning a desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

In certain embodiments, the template nucleic acid includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most poxvirus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO₄-TOPO, a complex of the topoisomerase covalently bound to the 3′ phosphate via a tyrosine in the topoisomerase (e.g., Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted often is combined with topoisomerase-reacted template and thereby incorporated into the template nucleic acid (e.g., http address www.invitrogen.com/downloads/F-13512_Topo_Flyer.pdf; http address at www.invitrogen.com/content/sfs/brochures/710_{—}021849%20_{—B}_TOPOCloning_bro.pdf; TOPO TA Cloning® Kit and Zero Blunt® TOPO® Cloning Kit product information).

A template nucleic acid sometimes contains one or more origin of replication (ORI) elements. In some embodiments, a template comprises two or more ORIs, where one functions efficiently in one organism (e.g., a bacterium) and another functions efficiently in another organism (e.g., a eukaryote). In some embodiments, an ORI may function efficiently in insect cells and another ORI may function efficiently in mammalian cells. A template nucleic acid also sometimes includes one or more transcription regulation sites.

A template nucleic acid often includes one or more selection elements. Selection elements often are utilized using known processes to determine whether a template nucleic acid is included in a cell. In some embodiments, a template nucleic acid includes two or more selection elements, where one functions efficiently in one organisms and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

The term “heterologous” as used herein refers to a nucleotide sequence from an organism species different than the organism species from which another nucleotide sequence originates (e.g., the translational enhancer sequence is from one organism species and the target ribonucleotide sequence is from another organism species). In some embodiments, the 5′ untranslated ribonucleotide sequence and/or translational enhancer sequence sometimes is heterologous to the target ribonucleotide sequence; the 5′ untranslated ribonucleotide sequence and/or translational enhancer sequence sometimes is heterologous to the cells from which the cell-free extract is derived; the promoter sometimes is heterologous to cells from which the cell-free extract is derived; the promoter sometimes is heterologous to the 5′ untranslated ribonucleotide sequence and/or translational enhancer sequence; the promoter sometimes is heterologous to the target ribonucleotide sequence; and/or the 5′ untranslated ribonucleotide sequence and/or translational enhancer sequence sometimes is from a virus that does not substantially infect cells from which the cell-free extract is derived. The term “homologous” as used herein refers to a nucleotide sequence from the same organism species from which another nucleotide sequence originates (e.g., the translational enhancer sequence and target ribonucleotide sequence are from the same organism species). In some embodiments, the 5′ untranslated ribonucleotide sequence and/or translational enhancer sequence sometimes is homologous to the target ribonucleotide sequence; the 5′ untranslated ribonucleotide sequence and/or translational enhancer sequence sometimes is homologous to the cells from which the cell-free extract is derived; the promoter sometimes is homologous to cells from which the cell-free extract is derived; the promoter sometimes is homologous to the 5′ untranslated ribonucleotide sequence and/or translational enhancer sequence; the promoter sometimes is homologous to the target ribonucleotide sequence; and/or the 5′ untranslated ribonucleotide sequence and/or translational enhancer sequence sometimes is from a virus that infects cells from which the cell-free extract is derived.

Certain nucleotide sequence sometimes are added to, modified or removed from one or more of the template nucleic acid elements, such as the promoter, 5′ UTR, target sequence, or 3′ UTR elements, to enhance or potentially enhance transcription and/or translation before or after such elements are incorporated in a template nucleic acid. In some embodiments, one or more of the following sequences may be modified or removed if they are present in a 5′ UTR: a sequence that forms a stable secondary structure (e.g., quadruplex structure or stem loop stem structure (e.g., EMBL sequences X12949, AF274954, AF139980, AF152961, S95936, U194144, AF116649 or substantially identical sequences that form such stem loop stem structures)); a translation initiation codon upstream of the target nucleotide sequence start codon; a stop codon upstream of the target nucleotide sequence translation initiation codon; an ORF upstream of the target nucleotide sequence translation initiation codon; an iron responsive element (IRE) or like sequence; and a 5′ terminal oligopyrimidine tract (TOP, e.g., consisting of 5-15 pyrimidines adjacent to the cap). A translational enhancer sequence and/or an internal ribosome entry site (IRES) sometimes is inserted into a 5′ UTR (e.g., EMBL nucleotide sequences J04513, X87949, M95825, M12783, AF025841, AF013263, AF006822, M17169, M13440, M22427, D14838 and M17446 and substantially identical nucleotide sequences). An AU-rich element (ARE, e.g., AUUUA repeats) and/or splicing junction that follows a non-sense codon sometimes is removed from or modified in a 3′ UTR. A polyadenosine tail sometimes is inserted into a 3′ UTR if none is present, sometimes is removed if it is present, and adenosine moieties sometimes are added to or removed from a polyadenosine tail present in a 3′ UTR. Thus, some embodiments are directed to a process comprising: determining whether any nucleotide sequences that reduce or potentially reduce translation efficiency are present in the elements, and removing or modifying one or more of such sequences if they are identified. Certain embodiments are directed to a process comprising: determining whether any nucleotide sequences that increase or potentially increase translation efficiency are not present in the elements, and incorporating such sequences into the template nucleic acid.

An ORF sometimes is mutated or modified (for example, by point mutation, deletion mutation, insertion mutation, and the like) to alter, enhance or increase, reduce, substantially reduce or eliminate the activity of the encoded protein or peptide. The protein or peptide encoded by a modified ORF sometimes is produced in a lower amount or may not be produced at detectable levels, and in other embodiments, the product or protein encoded by the modified ORF is produced at a higher level (e.g., codons sometimes are modified so they are compatible with tRNA in cells used to prepare a cell-free extract). To determine the relative activity, the activity from the product of the mutated ORF (or cell containing it) can be compared to the activity of the product or protein encoded by the unmodified ORF (or cell containing it).

A stop codon at the end of an ORF sometimes is modified to another stop codon, such as an amber stop codon described above. In some embodiments, a stop codon is introduced within an ORF, sometimes by insertion or mutation of an existing codon. An ORF comprising a modified terminal stop codon and/or internal stop codon often is translated in a system comprising a suppressor tRNA that recognizes the stop codon. An ORF comprising a stop codon sometimes is translated in a system comprising a suppressor tRNA that incorporates an unnatural amino acid during translation of the target protein or target peptide. Methods for incorporating unnatural amino acids into a target protein or peptide are known, which include, for example, processes utilizing a heterologous tRNA/synthetase pair, where the tRNA recognizes an amber stop codon and is loaded with an unnatural amino acid (e.g., http address www.iupac.org/news/prize/2003/wang.pdf). Unnatural amino acids include but are not limited to D-isomer amino acids, omithine, diaminobutyric acid, norleucine, pyrylalanine, thienylalanine, naphthylalanine and phenylglycine, alpha and alpha-disubstituted amino acids, N-alkyl amino acids, lactic acid, halide derivatives of natural amino acids such as trifluorotyrosine, p-Cl-phenylalanine, p-Br-phenylalanine, p-I-phenylalanine, L-allyl-glycine, beta-alanine, L-alpha-amino butyric acid, L-gamma-amino butyric acid, L-alpha-amino isobutyric acid, L-epsilon-amino caproic acid, 7-amino heptanoic acid, L-methionine sulfone, L-norleucine, L-norvaline, p-nitro-L-phenylalanine, L-hydroxyproline, L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminopropionic acid, L-Phe (4-benzyl), 2,4-diaminobutyric acid, 4-aminobutyric acid (gamma-Abu), 2-amino butyric acid (alpha-Abu), 6-amino hexanoic acid (epsilon-Ahx), 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, an amino acid derivitized with a heavy atom or heavy isotope (e.g., Au, deuterium, ¹⁵N; useful for synthesizing protein applicable to X-ray crystallographic structural analysis or nuclear magnetic resonance analysis), phenylglycine, cyclohexylalanine, fluoroamino acids, designer amino acids such as beta-methyl amino acids, Ca-methyl amino acids, Na-methyl amino acids, naphthyl alanine, and the like.

A template nucleic acid is of any form useful for in vitro transcription and/or translation. A nucleic acid sometimes is a plasmid, such as a supercoiled plasmid, sometimes is a linear nucleic acid (e.g., a linear nucleic acid produced by PCR or by restriction digest), sometimes is single-stranded and sometimes is double-stranded. A template nucleic acid for in vitro transcription and/or translation can be prepared by any suitable process. DNA templates sometimes are prepared by an amplification process, such as a polymerase chain reaction (PCR) process or transcription-mediated amplification process (TMA), and utilized in in vitro transcription systems and coupled in vitro transcription/translation systems, for example. In TMA, two enzymes are used in an isothermal reaction to produce amplification products detected by light emission (see, e.g., Biochemistry 1996 Jun. 25;35(25):8429-38 and http address www.devicelink.com/ivdt/archive/00/11/007.html). Standard PCR processes are known (e.g., U. S. Pat. Nos. 4,683,202; 4,683,195; 4,965,188; and 5,656,493, all herein incorporated by reference), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis. RNA templates utilized in non-coupled in vitro translation reactions often are transcribed from DNA in vitro, as described hereafter.

In Vitro Transcription

RNA is readily generated by contacting an RNA polymerase with a deoxyribonucleic acid template comprising an RNA polymerase promoter and a target ribonucleotide sequence. In some embodiments, the deoxyribonucleic acid template comprises a promoter for T7, T3, or SP6 RNA polymerase, each of which is commercially available. As an example, a DNA template with a T7 promoter is treated with T7 RNA polymerase according to manufacturers' specifications, and approximately 50 mRNA copies may be synthesized routinely for each DNA molecule in 30 minutes. The DNA template sometimes is degraded with an RNase-free DNase.

If some elements of a deoxyribonucleic acid template are RNA-based (e.g., nucleic acid in QB replicase system), a few RNA copies may be generated with T7 or other promoter system (e.g., Lizardi et al., “Exponential Amplification of Recombinant-RNA Hybridization Probes,” Bio/Technology 6:1197-1202, October 1988). Once RNA copies are generated a RNA-directed RNA polymerase is capable of generating a virtually unlimited number of copies of the RNA (one billion copies are easily attainable), and the diversity of any library remains the same. With RNA phages, such as QB, the library may be self-sustaining at the RNA level without the necessity of generating a DNA intermediate.

The 5′ end of each synthesized mRNA sometimes is modified by an exogenous cap, which can enhance translation by cell-free extracts generated from eukaryotic cells. In some embodiments, such as embodiments where transcription and translation are coupled, an mRNA is generated without a cap. 5′ capped mRNA may be generated in an in vitro transcription reaction (e.g., Hope and Struhl, Cell 43:177-188, 1985), in an in vitro translation process (Krieg and Melton, Nucleic Acids Res. 12:7057-7070, 1984) or in a coupled in vitro transcription/translation process, for example. Transcribed ribonucleic acids often are capped by including in the reaction mixture an excess of exogenous cap relative to a corresponding nucleotide (e.g., where a m⁷G(5′)pppG(5′), G(5′)pppG(5′), or other cap is coupled to an RNA transcript, it is added to the reaction mixture in excess of GTP). The ratio of cap to corresponding nucleotide often is about 4 to 1, and sometimes is about 3 to 1, about 2 to 1, about 5 to 1, about 6 to 1, about 7 to 1, about 8 to 1, about 9 to 1 or about 10 to 1. mRNA mapping kits are commercially available (e.g., Ambion (Texas)).

The cap sometimes comprises a methylated nucleoside base, sometimes is a dinucleoside triphosphate moiety, a (purine nucleoside)₂ triphosphate moiety or methylated moiety thereof, and sometimes is selected from the group of caps in Table 1.

TABLE 1

| | |
|---|---|
| m⁷G(5′)ppp(5′)G | (also referred to as m⁷Gp₃G) |
| m⁷(3′-O-methyl)(5′)Gppp(5′)G | (also referred to as m₂^{7,3′-O}Gp₃G) |
| A(5′)ppp(5′)G | (also referred to as Ap₃G) |
| m⁷G(5′)ppp(5′)A | (also referred to as m⁷Gp₃A) |
| b⁷Gp₃G | |
| e⁷Gp₃G | |
| m₂^{2,7}Gp₃G | |
| m₃^{2,2,7}Gp₃G | |
| m⁷Gp₃2′dG | |
| m⁷Gp₃m^{2′-O}G | |
| m⁷Gp₂m⁷G | |
| b⁷Gp₄G | |
| b⁷m^{3′-O}Gp₄G | |
| m₂^{2,7}Gp₄G | |
| m₃^{2,2,7}Gp₄G | |
| b⁷m²Gp₄G | |
| m⁷Gp₄m⁷G | |

In chemical formulas of Table 1, b is benzyl, e is ethyl, m is methyl, p is phosphate and d is deoxy. Alternative structural descriptions and methods for synthesizing such caps are known (e.g., Grudzien et al., RNA 10: 1479-1487 (2004)). Caps not including a 2′ or 3′ methoxy moiety in Table 1 sometimes are modified to include such a moiety, which can increase the efficiency with which the cap is correctly oriented in the capped mRNA product. A 2′ or 3′ methoxy moiety in a cap may be substituted with another moiety that cannot polymerize with a 3′ hydroxy group of another nucleotide (e.g., ethoxy).

An in vitro transcription and/or translation system generally comprises a cap transfer component (e.g., guanylyl transferase, adenylyl transferase, cap binding protein, a helicase and/or a methyltransferase), and sometimes comprises one or more components from a native cap transfer complex (e.g., an eIF4F complex, which includes eIF4A, eIF4E and eIF4G subunits). Such components sometimes are endogenous in the cell-free extract. Exogenous cap transfer components sometimes are added, where isolated components sometimes are added or components sometimes are expressed from a DNA or RNA encoding such a component.

In Vitro Translation

One or more cell-free extracts prepared from cells often are contacted with one or more template nucleic acids for synthesizing target protein or target peptide in in vitro translation reactions. A cell-free in vitro translation reaction sometimes is not coupled to an in vitro transcription reaction, and a ribonucleic acid that encodes a target protein or target peptide is contacted with a cell-free extract (e.g., Pelham et al., 1976, Eur. J. Biochem. 67: 247 and Roberts et al., 1973, Proc. Natl. Acad. Sci. USA 70: 2330). A cell-free in vitro translation reaction sometimes is coupled to an in vitro transcription reaction, and a deoxyribonucleic acid that encodes a target protein or target peptide often is contacted with a cell-free extract in such coupled systems (e.g., U.S. Pat. No. 5,492,817 incorporated by reference for all disclosure of translation systems).

In vitro translation systems, those that are coupled to transcription reactions and not coupled to transcription reactions, are known and commercially available, and many different types and systems are known and routinely used. An in vitro translation system sometimes comprises intact cells, and often comprises a cell-free extract with exogenous components. Examples of cell-free extracts commonly utilized for in vitro translation include rabbit reticulocyte lysates, wheat germ extracts and E. coli extracts. Such lysates and extracts can be prepared by the artisan using known procedures or are commercially available (Promega Corp.; Stratagene (California); Amersham (Illinois); Invitrogen (California) and GIBCO/BRL (New York)). In vitro translation systems generally contain endogenous and/or exogenous macromolecules such as enzymes; translation, initiation and elongation factors; chemical reagents; and ribosomes. Exogenous molecules can be supplied as isolated molecules as part of another cell-free extract in some embodiments. Mixtures of exogenous translation factors, as well as combinations of lysates or lysates supplemented with purified translation factors such as initiation factor-1 (IF-1), IF-2, IF-3 (alpha or beta), elongation factor T (EF-Tu) or termination factors, can be utilized for in vitro translation and coupled transcription/translation. Any appropriate template nucleic acid selected by the artisan can be utilized, such as a template nucleic acid described herein, and may be in any form suitable for cell-free in vitro translation. In some embodiments, the nucleic acid is in a supercoiled plasmid form or a linear form. Translation reactions generally contain a buffer such as Tris-HCl, HEPES, or other suitable buffering agent known to the artisan that maintains the solution at about pH 6 to about pH 8, and generally at about pH 7. A translation system sometimes includes one or more of the following components added in an appropriate amount known by the artisan: one or more reducing agents (e.g., dithiothreitol (DTT) or 2-mercaptoethanol); nucleotide triphosphates; one or more salts (e.g., potassium and magnesium containing salts; U.S. Pat. No. 5,492,817, incorporated by reference); one or more molecules that stimulate chain elongation (e.g., a polyamine such as spermidine at about 0.1 mM to about 1.0 mM); one or more energy sources (e.g., creatine phosphate and creatine kinase and others described hereafter); one or more fatty acids (e.g., myristic acid, palmitic acid, stearic acid, oleic acid, linoleic acid, linonenic acid, arachidonic acid, eicosapentaenoic acid), lipids, phospholipids, sphingolipids (e.g., phosphatidyl serine, phosphatidyl choline, phosphatidyl ethanolamine, phosphatidyl inositol, sphingosine, ceramide, sphingomyelin, cerbroside, ganglioside, glycosphingolipids) and/or cholesterol; one or more chaperone proteins (e.g., chaperonins from E. coli such as Hsp60, Hsp70, Skp, ClpB, FkpA, DsbC, Triger factor, foldases and the like); and any other components described hereafter in reference to a feeding solution.

An in vitro translation system also may include one or more components that inhibit one or more molecules that destabilize a DNA or RNA template nucleic acid or interfere with translation. Such molecules include, but are not limited to, one or more exonuclease inhibitors (e.g., inhibitors of exonuclease I, II, IIIm IVA, IVB, V, VII and VIII; Gam or Gam-like proteins), one or more endonuclease inhibitors (e.g., inhibitors of endonuclease I, III, IV, V, VII and VIII and vsr entodnuclease), one or more phosphatase inhibitors (e.g., pervanadate), one or more inhibitors of RecJ, dRpase, fpg, uvrABC, mutH, ruvC, ecoK, ecoB, mcrBC, mcrA, and/or mrr; one or more inhibitors of topoisomerase (e.g., inhibitors of topoisomerase I, II, III and IV), one or more inhibitors of translation termination factors (e.g., a RF1-like factor); and/or one or more polymerase inhibitors (e.g., inhibitors of DNA polymerase II and/or III). Inhibitors sometimes are small molecules, antibodies, protein or peptide binding partners, antisense. nucleic acids, siRNA, shRNA or ribozymes, for example. Some added components participate in translation, and include but are not limited to one or more tRNA molecules (e.g., tRNAs that recognize amino acid-encoding codons and suppressor tRNAs; commercially available or sometimes prepared from E. coli, yeast, calf liver or wheat germ); one or more ribosomes and ribosome components; one or more amino acids; an RNA polymerase; one or more deoxynucleotides; one or more caps; and one or more cap transferases. Protein components sometimes are provided to the system as exogenous protein (e.g., often an isolated protein) and sometimes are translated in situ from a DNA or RNA nucleic acid encoding the protein.

The translation process, including the movement of the ribosomes on RNA molecules, is inhibited at an appropriate time by the addition of an inhibitor of translation known to the artisan. In some embodiments, cycloheximide is added at a final concentration of about 1 μg/ml, or magnesium ion (e.g., MgCl₂) sometimes is added at a concentration of about 5 mM to maintain mRNA-80S ribosome-nascent polypeptide complexes (polysomes).

Optimal in vitro translation conditions can be determined and assessed. For example, translation of the target molecule can be monitored by a method known to the artisan during the in vitro process and/or after it is terminated, such as, for example, by detection by staining or autoradiography of translation products separated in gels, or by activity assays, including but not limited to CAT, luciferase, beta lactamase, or GUS assays, or by detection of fluorescent translation products (e.g., GFP), or by embodiments described hereafter.

Any type of appropriate cell selected by the artisan may be utilized to prepare a cell-free extract for in vitro translation. Cells sometimes are from immortalized cell lines, cells cultured as monolayers, cells cultured in suspension, and primary cell cultures. In some embodiments, cells utilized for preparing cell-free extracts include but are not limited to yeast cells (e.g., Saccharomyces cerevisiae cells and Pichia pastoris cells); insect cells (e.g., Drosophila (e.g., Drosophila melanogaster), Spodoptera (e.g., Spodoptera frugiperda Sf9 and Sf21 cells) and Trichoplusa (e.g., High-Five cells); nematode cells (e.g., C. elegans cells); avian cells (e.g., QT6 cells, QT-35 cells); amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells). Cells from insects, mammals (such as hamsters, mouse, rat, gerbil, porcine, bovine, monkey, and humans), for example, sometimes are utilized. These and other suitable host cells are available commercially, for example, from Invitrogen Corporation, (Carlsbad, Calif.), American Type Culture Collection (Manassas, Va.), and Agricultural Research Culture Collection (NRRL; Peoria, Ill.). Cell-free extracts sometimes are prepared from cells expressing one or more suppressor tRNAs, which are described above. Cell-free extracts often are prepared from cells capable of performing one or more post-translational modifications of interest. Post translational modifications include, but are not limited to, addition of a phosphoryl, alkyl (e.g., methyl), fatty acid (e.g., myristoyl or palmitoyl), isoprenyl, glycosyl (e.g., polysaccharide), acetyl or peptidyl (e.g., ubiquitin) moiety to a synthesized protein or peptide and proteolytic cleavage of a portion of the synthesized target protein or target peptide. A cell utilized for preparing a cell-free extract sometimes is deficient in one or more native components, such as components that reduce DNA or RNA stability or components that interfere with translation or detection of the target proteins or peptides, which are known to the artisan. Such components sometimes are reduced in cells by deleting or otherwise inactivating one or more genes or transcripts that encode a component. In some embodiments, the cells produce reduced amounts, non-detectable amounts or none of one or more of the following components: an exonuclease or endonuclease (e.g., an RNase such as RNase E, F, H, P and/or T; a DNase such as DNase I and/or II; a Rec protein; exonucleaseIII; exonuclease lambda; exonucleaseVII; endonuclease sl), topoisomerase and/or a component that binds to arsenic-containing agent (e.g., SlyD), for example (e.g., U.S. Patent application Publication no.20050136449, filed Oct. 1, 2004, entitled “Compositions and Methods for Synthesizing, Purifying, and Detecting Biomolecules”). Cell extracts sometimes are prepared from cells that express one or more suppressor tRNAs, such as a suppressor tRNA capable of loading any one of the twenty naturally occurring amino acids or an unnatural amino acid.

Any appropriate method for preparing a cell-free extract for use in in vitro translation reactions may be utilized. Cell-free extracts may be provided in any form, such as liquid form or solid form (e.g. frozen, desiccated or lyophilized), for example. An extract generally is a cell lysate or exudate typically processed to remove any intact cells and cellular debris. Cells can be disrupted by a variety of methods known to the artisan. Physical methods generally include osmotic shock, drying, shear forces (employing, for example, bead mills or blenders), temperature shock, ultrasonic disruption, or some combination of the above (e.g., a French press generates both shear forces and an explosive pressure drop). Other approaches combine chemical and physical methods of disruption and generally involve enzymatic (e.g., lysozyme) treatment followed by sonication or pressure treatment to maximize cell disruption. In some embodiments, cells are ruptured by mechanical shear and sometimes at a pressure of about 150 pounds per square inch or higher (e.g., French press).

The cells often are ruptured by mechanical shear, which can be applied by using a manually operated homogenizer, such as, for example, a Dounce homogenizer or a Potter Elvehjem homogenizer. In some embodiments, cells are ruptured using a device that forces cells in a chamber through an exhaust port having a relatively small cross-section under pressure (e.g., a French press or Emulsiflex high pressure cell homogenizer). Cells can be ruptured, for example, at a pressure of about 150 pounds per square inch or greater, about 175 pounds per square inch or greater, about 200 pounds per square inch or greater, about 500 pounds per square inch or greater, about 1,000 pounds per square inch or greater, about 5,000 pounds per square inch or greater, about 10,000 pounds per square inch or greater or about 20,000 pounds per square inch or greater. Any device and process for rupturing cells at such pressures can be utilized. Intact cells may be separated in cell lysates by any convenient method, and in some embodiments, centrifugation is utilized to clear intact cells to produce cell-free extracts. In certain embodiments, the supernatant of the clearing centrifugation is not subjected to column chromatography prior to use of the cell-free extract in translation reactions. In some exemplary embodiments of making a cell-free extract for translation, cells are lysed, preferably through mechanical shear forces, centrifugation is performed to remove intact cells from the lysate, and no further separation steps on the extract prior to using the extract in translation reactions.

Sedimentation is a common method for removing intact cells and cellular debris, and an appropriate centrifugation processes can by selected by the artisan to prepare the desired extract. Centrifugation processes include differential sedimentation and ultracentrifugation, sometimes are performed at centrifugal forces of about 100×g to about 300,000×g (e.g., about 2,000 rpm to about 100,000 rpm depending upon the rotor utilized), and sometimes involve the use of a sedimentation component, such as sucrose or cesium chloride, for example. In certain embodiments of preparing cell-free extracts for translation systems that have post-translational modification activity, such as but not limited to glycosylation activity, centrifugation is performed to remove unlysed cells and cell debris from the extract at forces of less than about 50,000×g, and preferably at forces of less than about 20,000×g.

Filtration, chromatography, or any other separation or purification procedures known to the artisan may be used to produce a desired extract. An extract often includes necessary components for synthesis that are not otherwise provided in the system. An extract can be concentrated using known concentration processes. Enzymes and other components present in the extract that provide energy and other components for the synthesis reaction can originate from the extracted cell or can be added during or after production of the extract. The term “cell-free extract” also includes a mixture of components crafted to imitate a cell lysate or exudate with respect to the components necessary or desired for protein or nucleic acid synthesis. A cell extract thus can be a mixture of components that imitates or improves upon a cell lysate or exudate in protein synthesis reactions and/or to provide components used for synthesis from a nucleic acid template. Such a mixture can be produced by obtaining a partial extract or fraction thereof and/or by mixing any number of individual components. In certain embodiments of preparing cell-free extracts for translation systems that have post-translational modification activity, such as but not limited to glycosylation activity, centrifugation is performed to remove unlysed cells and cell debris from the extract at forces of less than about 50,000×g, and preferably at forces of less than about 20,000×g, and no filtration or chromatography steps are performed in preparing the extract.

Cell-free extracts resulting from preparations described herein often comprise functional components for post-translational modification of synthesized target proteins or peptides, such as post-translational glycosylation. Cell-free extracts sometimes are treated with one or more nucleases that degrade endogenous DNA and/or RNA prior to contacting the extract with a template nucleic acid. Certain embodiments are directed to a process for preparing a cell-free extract from insect cells or mammalian cells, which comprises rupturing insect cells or mammalian cells at a pressure of 150 pounds per square inch or greater and separating (i.e., removing) intact cells, whereby a cell-free extract is prepared. Intact cells often are removed and separated by centrifugation or ultracentrifugation procedures known to the artisan. It is possible that not every intact cell is removed after separation, and often intact cells are substantially separated and substantially removed from the cell-free extract. A cell-free extract may be 90% or more free of intact cells, and often is 95% or more free of intact cells.

Some embodiments are directed to a process for preparing a cell-free extract for in vitro translation from insect cells, avian cells, or mammalian cells, comprising: rupturing insect cells or mammalian cells at a pressure of about 150 pounds per square inch or greater and removing intact cells, whereby a cell-free extract for in vitro translation is prepared. In some embodiments, the cells are cultured cells. In some embodiments, the cells are cultured mammalian cells. In some embodiments, the cells are cultured insect cells. Also featured is a process for preparing a cell-free extract from an insect cell or mammalian cell, which consists essentially of rupturing insect cells or mammalian cells and separating (i.e., removing) intact cells, whereby a cell-free extract is prepared. The term “consists essentially of” as applicable to the previously described embodiment refers to a process in which a cell-free extract is prepared by rupturing cells and removing intact cells without performing additional preparative steps that separate protein or membrane components from the extract (e.g., not subjecting the extract to size-exclusion chromatography). Such processes can include other steps, however, such as adjusting pH, adding or subtracting salts and/or buffers (e.g., dialyzing the extract) and adding components useful for in vitro translation (e.g., adding an energy source). In some embodiments, the cells are cultured cells. In some embodiments, the cells are cultured cells, and the resulting extract has endogenous functional glycosylation components. In some embodiments, the cells are cultured cells, and the resulting extract has endogenous functional signal sequence processing components. In some embodiments, the cells are cultured cells. In some embodiments, the cells are cultured insect cells. In some embodiments, the cells are cultured mammalian cells.

Also provided is a process for synthesizing a target protein or target peptide in a cell-free system, comprising: contacting a nucleic acid that encodes a target protein or target peptide with a cell-free extract, where the cell-free extract is prepared by a process which comprises rupturing insect cells, avian cells, or mammalian cells at a pressure of about 150 pounds per square inch or greater and separating intact cells, whereby the target protein or target peptide is synthesized. Also featured is a process for synthesizing a target protein or target peptide in a cell-free system, comprising: contacting a nucleic acid that encodes a target protein or target peptide with a cell-free extract, where the cell-free extract is prepared by a process which consists essentially of rupturing insect cells, avian cells, or mammalian cells and separating intact cells, whereby the target protein or target peptide is synthesized.

Some embodiments are directed to a process for synthesizing a glycosylated target protein or target peptide in a cell-free system, comprising: contacting a nucleic acid that encodes a target protein or target peptide with a cell-free extract, where the cell-free extract is prepared by a process which comprises rupturing insect cells or mammalian cells at a pressure of about 150 pounds per square inch or greater and separating intact cells, whereby glycosylated target protein or target peptide is synthesized. Also, some embodiments are directed to a process for synthesizing a glycosylated target protein or target peptide in a cell-free system, comprising: contacting a nucleic acid that encodes a target protein or target peptide with a cell-free extract, where the cell-free extract is prepared by a process which consists essentially of rupturing insect cells or mammalian cells and separating intact cells, whereby glycosylated target protein or target peptide is synthesized. Multiple glycosidic linkages are known to the artisan, including but not limited to N-glycosidic linkages (e.g., GlcNAc-B-Asn, Glc-β-Asn, Rha-Asn and Glc-β-Arg linkages); β-glycosidic linkages (e.g., linkages to Ser, Thr, Tyr, Hyp [hydroxyproline], and Hyl [hydroxylysine]; GalNAc-Ser/Thr, GalNAc-β-Ser/Thr, Gal-Ser/Thr, Man-Ser/Thr, Fuc-Ser/Thr, Glc-β-Ser, Pse-Ser/Thr, DiActrideoxyhexose-Ser/Thr, FucNAc-β-Ser/Thr, Xyl-β-Ser, Glc-Thr, GlcNAc-Thr, Gal-β-Hyl,Gal-Hyp, Gal-β-Hyp, Ara-Hyp Ara-β-Hyp, GlcNAc-Hyp, Glc-Tyr and Glc-β-Tyr linkages); C-mannosyl linkages (e.g., mannosyl linkage to C-2 of the Trp through a C—C bond); phosphoglycosyl linkages (e.g., attachment of sugar (e.g., GlcNAc, Man, Xyl, and Fuc) to protein via a phosphodiester bond; GlcNAc-1-P-Ser, Man-1-P-Ser, Xyl-1-P-Ser, Fuc-β-1-P-Ser linkages); and glypiated linkages (e.g., Man is linked to phosphoethanolamine, which in turn is attached to the terminal carboxyl group of a protein). Extent of glycosylation can be assessed by the artisan using known methods (e.g., Spiro, Glycobiology 12: 43R-56R (2002)) and as those described herein. In certain embodiments, a cell-free extract is derived from cultured cells. In certain embodiments, a cell-free extract is derived from cultured mammalian cells. In certain embodiments, a cell-free extract is derived from cultured cells. In certain embodiments, a cell-free extract is derived from cultured insect cells. In certain embodiments, a cell-free extract is derived from cultured avian cells.

In certain embodiments, a cell-free extract is contacted with a deoxyribonucleic acid in a coupled in vitro transcription/translation reaction. A template deoxyribonucleic acid described herein sometimes is utilized. In some embodiments, a cell-free extract is contacted with a ribonucleic acid in an in vitro translation reaction. A template ribonucleic acid described herein sometimes is utilized, and the ribonucleic acid sometimes comprises a cap, such as a cap described herein. The template ribonucleic acid sometime comprises a translational enhancer sequence, such as the tobacco mosaic virus omega sequence, or substantially homologous sequences, or translation-enhancing sequences that have one or more poly (CAA) sequences, as described herein.

In vitro translation systems often include one or more energy sources, which sometimes are provided in a “feeding solution”. When included in a feeding solution, energy sources can be contacted with in vitro translation reactants in a variety of manners known to the artisan and as described herein. A feeding solution sometimes contains one or more of the following components: one or more buffers (10-100 mM); one or more salts; one or more reducing agents; one or more energy sources and/or cofactors; four or more amino acids; and sometimes ammonium acetate at about 80 mM. Any suitable buffer can be utilized, such as 50 mM HEPES, for example. The pH of the feeding solution sometimes is higher than the pH of the reaction system (e.g., the feeding solution may be at about pH 8.0 and the reaction system may be at about pH 7.6). Buffers and salts included in the feeding solution often are identical to those in the initial reaction to maintain ionic strength, and sometimes calcium is added to a feeding solution (e.g., 2 mM calcium chloride sometimes is added to the feeding solution). Any suitable salt may be utilized, such as a magnesium containing salt (e.g., MgCl₂ at a concentration of about 5 mM-50 mM or about 1 OmM to about 15 mM), potassium glutamate (e.g., about 180 mM to about 250 mM, about 230 mM), and/or CaCl₂ (e.g., about 1 mM to about 750 mM, or about 5, 10, 20, 30, 50 or 100 mM). Any suitable reducing agent may be utilized, such as dithiothreitol and/or beta-mercaptoethanol. Any suitable energy source and/or cofactor may be utilized. An energy source sometimes comprises one or more phosphate containing agents, such as adenosine triphosphate, guanosine triphosphate, glycolysis intermediates (e.g., glucose-6-phosphate, 3-phosphoglycerate, phosphoenol pyruvate, acetyl phosphate, phosphopyruvate, fructose-6-phosphate, glyceraldehydes-3-phosphate), carbamoyl phosphate and/or creatine phosphate, for example, which sometimes are utilized at a concentration of about 1 mM to about 200 mM, about 10 mM to about 100 mM, about 20 mM to about 60 mM and about 80 mM or less (e.g., U.S. Pat. No. 6,337,191; Swartz, et al. Jan. 8, 2002. “In vitro Protein Synthesis using Glycolytic Intermediates as an Energy Source). An energy source sometimes is a molecule involved in cell metabolic pathways, such as a saccharide (e.g., glucose), an oligosaccharide (e.g., sucrose) or pyruvate. A cofactor sometimes is niacin, nicotinamide adenine dinucleotide (NAD⁺ or NADH), riboflavin, flavin adenine dinucleotide (FAD or FADH), pantothenic acid, coenzyme A (CoA), vitamin B-12, coenzyme B-12, thiamin (B-1), thiaminpyrophosphate (TPP) and/or folate, for example, sometimes utilized at a concentration of about 0.1 mM to about 25 mM or about 0.1 mM to about 1 mM. Any amino acid may be utilized, sometimes at a concentration of about 0.05 mM to about 5.0 mM, or about 0.25 to about 2.5 mM. In an embodiment, a feeding solution comprises (final concentrations) 57.5 mM HEPES-KOH pH 8.0, 1.7 mM M DTT, 230 mM potassium glutamate, 12.5 mM MgOAc, 80 mM NH₄OAc, 2 mM CaCl₂, 30 mM Glu-6-P, 0.3 mM NAD, 34 mM folinic acid, 0.35 mM cAMP and molecular biology grade water.

Coupled and non-coupled cell-free in vitro translation processes can be performed in a variety of systems, including but not limited to, batch systems, feeding/dilution systems or continuous flow systems, bilayer overlay systems and continuous exchange systems. Incubation times vary significantly with the volume of the translation mix and the temperature of the incubation, and are appropriately selected by the artisan. Incubation temperatures can be between about 4° C. to about 60° C., generally about 15° C. to about 50° C., sometimes about 25° C. to about 45° C., and often about 25° C. to about 37° C.

A batch reaction generally is performed in a closed system, in which there is fast initial rate of synthesis that slows and eventually stops after about 3 hours. Incubation times range from about 5 minutes to many hours, and generally are about thirty minutes to five hours, usually about one to three hours. The composition of the reaction mix changes as amino acids are incorporated or metabolized, and energy sources are metabolized, generating inhibitory free phosphate. A batch reaction often includes one or more energy sources. Sometimes a batch system is spiked one or more times during translation with one or more energy sources or feeding solutions. Examples of batch systems are known (e.g., Kawasaki et al. (1995), “A Long-Lived Batch Reaction System of Cell-Free Protein Synthesis.” Analytical Biochemistry, vol. 226:320-324; Patnaik et al. (1998), “E. coli-Based In vitro Transcription/Translation: In Vivo-Specific Synthesis Rates and High Yields in a Batch System.” BioTechniques, vol. 24:862-868; and Kigawa et al. (1991)).

In continuous flow systems, fresh components are supplied to a reaction chamber continuously over time. A feeding solution sometimes is continuously flowed into a reaction vessel in such systems. This continuous feed approach provides a continuous supply of reactants, energy sources and cofactors, and also removes inhibitory products and by-products. Examples of continuous flow systems are known (e.g., U.S. Pat. No. 5,478,730 (Alakhov et al., 1995); U.S. Pat. No. 5,593,856; JP Patent 10080295; “Production of an Enzymatic Active protein Using a Continuous Flow Cell-Free Translation System.” Journal of Biotechnology, vol. 25:221-230; and Spirin et al. (1988), “A Continuous Cell-Free Translation System Capable of Producing Polypeptides in High Yield.” Science, vol. 24:1162-1164). All or these references are herein incorporated by reference for disclosure of in vitro translation systems.

In bilayer overlay systems, a high density reaction mix is overlaid with a feeding solution and components are exchanged through passive diffusion. The reaction rate can be slowed by not shaking the reaction vessel. Examples of such systems are known (e.g., Sawasaki et al., A bilayer cell-free protein synthesis system for high-throughput screening of gene products. 2 FEBS Lett. 6;514(1):102-5. 2002).

In continuous exchange systems, the reaction chamber is separated from a feeding solution by one or more dialysis membranes, allowing constant exchange of substrates and by-products. Such a system sometimes is a dialysis bag containing in vitro synthesis reactants in a container comprising a feeder solution. Examples of such systems are known (e.g., U.S. Pat. No. 5,478,730 (Alakhov et al., 1995); Endo et al. (1992); Davis. et al., “Large Scale Dialysis Reactions Using E. coli S30 Extract Systems,” Promega Notes 56 (1996; p. 14-21)).

Certain embodiments are directed to a process for cell-free translation of a protein or peptide, comprising: contacting in a system a cell-free extract derived from mammalian cells, avian cells, or insect cells with a ribonucleic acid comprising a 5′ untranslated region comprising an translational enhancer ribonucleotide sequence and a target ribonucleotide sequence, whereby a protein or peptide is translated from the target ribonucleotide sequence. In some embodiments, the ribonucleic acid comprises an exogenous cap. In exemplary embodiments, the cell-free extract is derived from cultured mammalian cells or insect cells. In some embodiments, the translational enhancer is the TMV omega sequence (SEQ ID NO:1), or a sequence substantially homologous to the TMV omega sequence that has translation-enhancing activity, or a TMV-related sequence having at least one poly (CAA) sequence that has translation enhancing activity. In some embodiments, the cell extract is derived from cultured cells. In some embodiments, the cell extract is derived from cultured avian cells. In some embodiments, the cell extract is derived from cultured mammalian cells

Also provided is a process for cell-free translation of a protein or peptide, comprising: contacting in a system a cell-free extract derived from mammalian cells or insect cells with a deoxyribonucleic acid that encodes a ribonucleic acid which comprises a 5′ untranslated region comprising an translational enhancer ribonucleotide sequence and a target ribonucleotide sequence, whereby a protein or peptide is translated from the target ribonucleotide sequence. In some embodiments, the ribonucleic acid is contacted with an exogenous cap, such as a cap described herein, resulting in a capped nucleic acid. In certain embodiments, the protein or peptide translated from the target ribonucleotide sequence is post-translationally modified, and sometimes is glycosylated. A template nucleic acid as described herein is contacted with the cell-free lysate. The system sometimes is contacted with an energy source one or more times, and sometimes the system is in contact with one or more devices comprising a semi-permeable membrane, such as a dialysis membrane.

In Vivo Transcription and Translation

Template nucleic acids described herein may be utilized to produce products encoded therefrom in cells. For example, some embodiments are directed to a process for synthesizing a protein or peptide in non-plant cells, which comprises maintaining or growing non-plant cells comprising a ribonucleic acid which comprises a 5′ untranslated region comprising an translational enhancer ribonucleotide sequence and a target ribonucleotide sequence, whereby a protein or peptide encoded by the target ribonucleotide sequence is synthesized from the ribonucleic acid. In some embodiments, the ribonucleic acid is contacted with an exogenous cap, such as a cap described herein, thereby resulting in a capped nucleic acid. The ribonucleic acid sometimes is translocated into the cells (e.g., transfected) using any pertinent translocation process (e.g., using calcium phosphate, electroporation and/or Lipofectin®), and the ribonucleic acid sometimes is transcribed from a deoxyribonucleic acid translocated into the cells that encodes the translational enhancer ribonucleotide sequence and the target ribonucleotide sequence.

Also provided is a process for synthesizing a ribonucleic acid in cells, comprising: maintaining or growing non-plant eukaryotic cells comprising a deoxyribonucleic acid encoding a ribonucleic acid comprising a 5′ untranslated region comprising an translational enhancer ribonucleotide sequence and a target ribonucleotide sequence under conditions suitable for ribonucleic acid synthesis; and contacting the cells with an exogenous cap; whereby a ribonucleic acid is synthesized that comprises the cap, the translational enhancer ribonucleotide sequence and the target ribonucleotide sequence. The cap may be contacted with the cells at any time, such as before, during or after synthesis of the ribonucleic acid or protein. The cap sometimes is added to the cell culture media, and sometimes it is incorporated into the cells by a translocation method (e.g., electroporation).

In the foregoing processes, the non-plant cell sometimes is a mammalian cell, an avian cell, or an insect cell. For example, a cell can be a hamster cell (e.g., a Chinese hamster ovary cell, a Baby hamster kidney cell), a monkey cell (e.g., a COS cell), a human cell (e.g., a HeLa cell), and avian cell (e.g., a QT 6 cell or a QT-35 cell), or an insect cell (e.g., Spodoptera, Drosophila). Any suitable type of such cell selected by the artisan can be utilized, such as a cell from primary culture, an immortalized cell line, a plated cell culture or a cell culture in suspension, for example. The nucleic acid utilized sometimes is a template nucleic acid described herein.

Methods for expressing target proteins and target peptides in host cells are well known. See, e.g., Hodgson, Expression Systems: A User's Guide Bio/Technology 11, 887-893, 1993. Typical host cells include insect cells (See, e.g., Luckow et al., Bio/Technology (1988) 6: 47-55, 1988; Baculovirus Expression Vectors: A Laboratory Manual, O'Rielly et al. (Eds.), W. H. Freeman and Company, New York, 1992; and U.S. Pat. No. 4,879,236; bacteria (see, e.g, Sambrook, et al., Molecular Cloning: A Laboratory Manual, Cold Spring Harbor Laboratory, Cold Spring Harbor, N.Y., 1989; Gene Expression in Recombinant Microorganisms, A. Smith, ed., Marcel Dekker, Inc. New York, 1994); yeast (See, e.g., Pichia Protocols, in Methods in Molecular Biology, vol. 103, Higgins and Cregg, eds., Humana Press, NJ, 1998); and plant cells (see, e.g, Kusnadi et al., Production of Recombinant Proteins in Transgenic Plants: Practical Considerations, Biotech. and Bioeng. 5: 473-84, 1997). The foregoing lists are exemplary and are not meant to be limiting. Expressed target proteins and target peptides may be isolated and/or purified according to processes described herein.

Post Transcription and Translation Processes

Translated target products sometimes are isolated or purified after transcription and/or translation. The term “isolated” as used herein refers to material removed from its original environment (e.g., the natural environment if it is naturally occurring, or a host cell if expressed exogenously), and thus is altered “by the hand of man” from its original environment. The term “purified” as used herein with reference to molecules does not refer to absolute purity. Rather, “purified” is intended to refer to a substance in a composition that, if a polypeptide, contains fewer polypeptide species other than the polypeptide of interest in comparison to the organism from which it originated. “Purified,” if a nucleic acid, refers to a substance in a composition that contains fewer nucleic acid species other than the nucleic acid of interest in comparison to the organism from which it originated. Sometimes, a polypeptide or nucleic acid is “substantially pure,” indicating that the polypeptide or nucleic acid represents at least 50% of polypeptide or nucleic acid on a mass basis of the composition. Often, a substantially pure polypeptide or nucleic acid is at least 75% on a mass basis of the composition, and most sometimes at least 95% on a mass basis of the composition.

A transcribed nucleic acid, for example, may be isolated by ethanol precipitation and optionally processed further. Target proteins or target peptides may be purified according to their inherent properties (e.g., the protein or peptide is charged, hydrophobic and/or specifically binds to a ligand) as known in the art. A target protein or target peptide translated with a tag can be isolated by contacting the tag with a specific binding agent. Such binding agents are known and described herein, and often are linked to a solid support. The term “solid support” or “solid phase” as used herein refers to a wide variety of materials including solids, semi-solids, gels, films, membranes, meshes, felts, composites, particles, and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Diamandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997 (1998); Kessler et al., Agnew. Chem. Int. Ed. 40: 165 (2001); Smith et al., J. Comb. Med. 1: 326 (1999); Orain et al., Tetrahedron Lett. 42: 515 (2001); Papanikos et al., J. Am. Chem. Soc. 123: 2176 (2001); Gottschling et al., Bioorg. And Medicinal Chem. Lett. 11: 2997 (2001). For example, target proteins and target peptides sometimes are purified by a polyhistidine tag-chelating resin (e.g., ProBond™ purification system (Invitrogen, California)) and/or a cysteine-rich tag purification resin (e.g., Lumio® agent (Invitrogen, California) linked to a solid phase). Featured herein are isolated or separated nucleic acids, proteins or peptides prepared by transcription and/or translation systems described herein. Provided also are arrays comprising one or more, two or more, three or more, etc., of the proteins or peptides produced by the methods of the present invention, where the synthesized proteins and/or peptides are immobilized at discrete sites on a solid support in an ordered array. Such arrays sometimes are high-density arrays, such as arrays in which each spot comprises at least 100 protein molecules per square centimeter. Solid supports include but are not limited to a glass slide, a microchip, a microtiter plate, a chromatography support, a nanotube, and the like.

In some embodiments, one or more detergents, lipids, liposomes or nanodiscs (see, for example, U.S. Patent Application Publication No. 2005/0182243, 2005/0152984, 2004/0053384, or WO 02/040501, herein incorporated by reference for all disclosure of nanodiscs and scaffold proteins) are added to an in vitro transcription and/or translation system before, during or after translation. A detergent can aid in solublization of a target protein for further processing (e.g., protein isolation). Detergents include, but are not limited to, a detergent described above, anionic detergents such as sodium n-dodecyl sulfate (SDS); dihydroxy or trihydroxy bile acids (and their salts), such as cholic acid (sodium cholate), deoxycholic acid (sodium deoxycholate), taurodeoxycholic acid (sodium taurodeoxycholate), taurocholic acid (sodium taurocholate), glycodeoxycholic acid (sodium glycodeoxycholate), glycocholic acid (sodium glycocholate); cationic detergents such as cetyl trimethyl-ammonium bromide (CTAB); non-ionic detergents such as the polyoxyethylenes NP-40, TRITON® X-100, TRITON® X-114, C₁₂E₈, C₁₂E₉, GENAPOL® X-080, GENAPOL® X-100, LUBROL® PX, BRIJ® 35, TWEEN® 20, and TWEEN® 20; alkyl glycosides such as dodecyl-β-D-maltoside (“dodecyl maltoside”), n-nonyl-β-D-glucopyranoside, n-octyl-β-D-glucopyranoside (“octyl glucoside”), n-heptyl-β-D-glucopyranoside, and n-hexyl-β-D-glucopyranoside; alkylamine oxides such as lauryl dimethylamine oxide (LDAO); and zwitterionic detergents, such as CHAPS, CHAPSO, n-dodecyl-N,N-dimethylglycine, and ZWITTERGENTS® 3-08, 3-10, 3-12, 3-14, and 3-16.

Overexpression of proteins in cells sometimes leads to the production of insoluble aggregates of misfolded proteins in inclusion bodies. Often considered a nuisance, the formation of inclusion bodies has the advantage. of a high enrichment of the desired protein at an early stage of purification. Furthermore, the recombinant protein is protected in inclusion bodies against proteolysis by intracellular proteases. These inclusion bodies can easily be purified and may be the best method for the production of proteins that are lethal to the host cells. However, the solubilization of the expressed protein often is obtained using strongly denaturing conditions. Since inclusion body proteins do not readily disintegrate under physiological conditions, the solubilization requires rather strong chaotropic agents such as 6 M guanidine hydrochloride or 6M-8 M urea. Guanidine hydrochloride often is preferred over urea because it may solubilize extremely sturdy inclusion bodies, and because urea solutions may contain isocyanate leading to carbamylation of the free amino groups of the polypeptide. In the case of proteins containing cysteine, the isolated inclusion bodies usually contain some interchain disulfide bonds which reduce the solubility. Addition of reducing agents, like DTT and/or 2-mercaptoethanol, in combination with chaotropic agents allows reduction of the interchain disulfide bonds.

As described above, translation efficiency can be monitored during and/or after translation by known procedures. In such methods, the amount of target protein or peptide often is determined. In some embodiments, translation of a target protein or target peptide is assessed by mass spectrometric analysis (e.g., U.S. Pat. No. 6,322,970, herein incorporated by reference for all disclosure of mass spectrometric analysis of proteins): In certain embodiments, a labeled amino acid such as ³⁵S-methionine can be included in the translation reaction together with an amino acid mixture having a full complement of amino acids or lacking the unlabeled amino acid counterpart (e.g., methionine). A labeled non-radioactive amino acid may be incorporated into a nascent polypeptide in certain embodiments. For example, the translation reaction can contain a mis-aminoacylated tRNA (U.S. Pat. No. 5,643,722, herein incorporated by reference for all disclosure of translation reactions and tRNA incorporation and misincorporation). The system generally is incubated to incorporate the non-radioactive marker into the nascent polypeptide and polypeptides containing the marker can be detected using a detection method appropriate for the marker known to the artisan. Mis-aminoacylation of a tRNA molecule also can be used to add a marker to the polypeptide to facilitate isolation of the polypeptide. Such markers include, for example, biotin, streptavidin and derivatives thereof (e.g., U.S. Pat. No. 5,643,722, herein incorporated by reference). In some embodiments, tagged target proteins or peptides are detected by detectable molecules that specifically interact with the tag, as known by the artisan (e.g., InVision® His-tag detection and Lumio® detection of cysteine-rich tags (Invitrogen, California)). In certain embodiments, the artisan monitors a known function of the translated target protein or target peptide using an appropriate assay known in the art, which is useful for monitoring the amount of functional target protein or peptide translated. In some embodiments, efficiency or amounts of post-translational modification of a target protein or peptide sometimes is monitored by a method known to the artisan (post-translational modifications are described herein). For example, a target protein or peptide sometimes is monitored by electrophoresis (e.g., a post-translationally modified product often migrates at a lower molecular weight (e.g., proteolytically processed product) or higher molecular weight (e.g., a glycosylated or ubiquinated product) than unmodified product). A product sometimes is contacted with an antibody or enzyme that specifically binds to or degrades, respectively, the post translational modification (e.g., an antibody or glycosidase (e.g., β-3-N-acetylglucosaminidase) that specifically binds to or degrades a polysaccharide linked to a protein).

Transcription products also may be monitored and isolated. For example, relative amounts of a target ribonucleic acid (e.g., an untranslated or translated ribonucleic acid), capping efficiency and presence and/or degree of modification (e.g., methylation, acetylation) sometimes are determined by methods known to the artisan.

Kits

Kits comprise one or more containers, which contain one or more of the compositions and/or components described herein. A kit comprises one or more of the components in any number of separate containers, packets, tubes, vials, microtiter plates and the like, or the components may be combined in various combinations in such containers. In some embodiments, a kit comprises a container that includes a cell-free extract derived from mammalian cells (e.g., human (e.g., HeLa cells), hamster (e.g., Chinese Hamster Ovary cells, Baby Hamster Kidney cells)) or insect cells (e.g., Spodoptera, Drosophila), prepared by a process described herein, in which cells are lysed by mechanical shear forces and chromatography separation steps are not performed in preparing the cell extract. Exemplary kits include extracts made from insect cells and one or more buffers, energy sources, energy generating enzymes, amino acids, inhibitors, or enzymes for use in an in vitro translation reaction. A kit sometimes comprises a container that includes a template nucleic acid described herein.

One or more of the following components may be included in a kit: one or more nuclease inhibitors (e.g., a RNase or DNase inhibitor); one or more phosphatase inhibitors; one or more polymerase inhibitors; one or more nucleotides or derivatives thereof; one or more amino acids or derivatives thereof; one or more polymerases (e.g., an RNA polymerase); one or more proteins that stabilize a deoxyribonucleic acid or ribonucleic acid (e.g., Gam protein); one or more ribosome proteins; one or more cap transfer proteins; a nucleic acid that encodes one or more of the proteins described herein, proteins necessary for in vitro synthesis and/or proteins that enhance in vitro synthesis of a biomolecule; one or more caps (e.g., a cap in Table 1); one or more cofactors; one or more buffers or buffer salts; one or more energy sources (e.g., containing ATP and/or creatine phosphate); one or more nucleic acid templates (e.g., a nucleic acid template described herein); one or more non-plant cells from an organism species (e.g., hamster, human, insect); one or more oligonucleotide primers; one or more reverse transcriptases; one or more recombination proteins; one or more topoisomerases; one or more detergents, one or more restriction endonucleases; one or more ligases; one or more terminating agents (e.g., ddNTPs); one or more transfection reagents; pyrophosphatase; one or more RNA or protein purification components (e.g., solid phase derivitized with a molecule that specifically binds a tag on a synthesized protein); one or more reagents that bind to the synthesized protein or RNA or a tag thereof (e.g., an arsenic containing detection agent that specifically binds to a cysteine-rich tag of a synthesized protein); one or more reagents to determine the efficiency of the kit or assay for production of RNA or protein products; and the like. A kit sometimes comprises a component for performing in vitro transcription and/or translation, such as a continuous flow reaction component or a continuous exchange reaction component (e.g., a dialysis membrane).

A kit sometimes is utilized in conjunction with process described herein, and sometimes includes instructions for performing one or more processes described herein and/or a description of one or more compositions described herein. Instructions and/or descriptions may be in printed form and may be included in a kit insert. A kit also may include a written description of an intemet location that provides such instructions or descriptions.

### EXAMPLES

The examples set forth below illustrate but do not limit the invention.

### Example 1

### Cell-Free Extracts with Post-Translational Modification Activity

One liter of Spodoptera frugiperda 21 (Sf21) cells was harvested at a density of approximately 1.5×10⁶ cells/ml, washed two or three times with buffer A (40 mM Hepes-KOH, pH 8, 100 mM KOAc, 1 mM Mg(OAc)₂, 2 mM CaCl₂, 4 mM dithiothreitol (DTT)). The pellet was resuspended in a half volume of buffer.

Cell-free extracts were prepared using two different procedures. In one procedure, a Mini-Bomb cell disruption chamber (KONTES Glass Company, Vineland, N.J.) was loaded with the washed cells. The cell suspension was pressurized and equilibrated for 30 min at a nitrogen pressure of 120 psi, and cells were then disrupted to flow under atmospheric pressure (Crude Fraction). The crude fraction was centrifuged for 15 min at 14,000 rpm at 4° C. in a microcentrifuge. The supernatant (cleared fraction) was passed through a Sephadex G-25 column equilibrated with buffer B (40 mM Hepes-KOH, pH 8, 100 mM KOAc, 5 mM Mg(OAc)₂, 4 mM DTT). Fractions eluting from the column with the highest RNA/protein concentration were pooled, aliquoted, and stored at −70° C. (column fraction). In an alternate procedure, resuspended cells were lysed by a single passage through a French Press at a pressure of 500 psi and cleared by centrifugation at 14000×g for 15 min at 4° C., aliquoted into small tubes and rapidly stored at −80° C. The final samples had a concentration titer of 100 OD₂₆₀ units or higher.

Expression of genes (gp120 and DHFR) was driven by the T7 promoter. An omega 5′ UTR from Tobacco Mosaic Virus was located upstream of the mentioned genes. In vitro transcription was performed using a commercially available transcription kit (mMessage mMachine T7 Ultra (Ambion)). The resulting capped transcripts were purified by LiCl precipitation and a single ethanol wash.

Translation reactions were performed in a total volume of 25 μl in the presence of the following reagents: 8 μl cell-free extract, 30 mM Hepes-KOH, pH 8, 1.6 mM Mg(OAc)₂, 100 mM KOAc, 2.5 mM DTT, 0.25 mM spermidine, 1.75 mM ATP, 0.25 mM GTP, 10 mM creatine phosphate, 25 μM amino acids, 2.5 μg mRNA, 1 mg/ml of creatine kinase, and 2 pmol of [³⁵S]methionine (10 μCi/μl, 1175 Ci/mmol) for trace labeling purposes. The reaction proceeded for 2 hours at 25° C. After translation, fractions of the reaction mix were treated with Endoglycosidase F (PNGase F, New England Biolabs). This enzyme cleaves between the innermost GlcNAc and asparagine residues of high mannose, hybrid, and complex oligosaccharides from N-linked glycoproteins. Proteins were resolved by SDS-PAGE and the gel exposed overnight to an X-ray film. Translational efficiency of the cell-free extract then was assessed.

Cell-free extracts prepared by the two procedures were used in otherwise identical translation reactions that employed the same amounts of RNAs coding for the proteins gp120 and DHFR. Bands of comparable intensity were obtained for each protein regardless of the extract preparation strategy employed. Treatment of the products with endoglycosidase F (PNGase F) permitted the distinction between glycosylated and unglycosylated gp120. The gel-filtration step used in one method of extract preparation abolished glycosylation activity, and it was determined that clarification of the crude extract by centrifugation after lysis was sufficient for obtaining an extract for translation having glycosylation activity.

### Example 2

### Demonstration of Signal Sequence Cleavage and Glycosylation in Translation System

Briefly, extracts of cells (Spodoptera frugiperda 21) were produced by the method of Example 1 that used a French Pressure Cell for lysis, and did not include a column chromatography step. Typically not more than 8% of the cells were lysed prior to passage through the French pressure cell. After passage through the French pressure cell, typically more than 99% of the cells were lysed. The lysate was cleared by centrifugation at 14,000×g for 15 min at 4° C., aliquoted into small tubes and rapidly stored at −80° C. The samples typically had a concentration titer of 100 OD₂₆₀ units or higher.

For evaluation of the glycosylation of proteins translated in the insect cell free system, the HIV-1 gp120protein (as a known glycoprotein control) and six ORF-encoded proteins with predicted N-linked glycosylation sites (PGS) and at least one predicted transmembrane site (PTMS) serving as signal sequence (Table 2) were expressed using the conditions described above. The six human ORFs were from the Invitrogen Ultimate™ ORF Clone collection (Invitrogen Corp, Carlsbad, Calif.). The ORFs were recombined into a variant of vector pEU3NII (Invitrotech, Japan) that has the omega sequence of the tobacco mosaic virus downstream of a T7 promoter and that was previously adapted to the Gateway technology (Invitrogen, Carlsbad, Calif.). Genes were transcribed using the Ambion's mMESSAGE mMACHINE T7 Ultra (Ambion, Austin, Tex.) following the manufacturer's directions.

TABLE 2
Genes Used in Translation System

| Clone Name | Genbank | | | |
|---|---|---|---|---|
| or ID No. | accession No. | MW (kDa) | TMPS | PGS |
| Gp120 | AAR05834 | 57.7 | 1 | 23 |
| IOH 12272 | BC010957 | 13 | 1 | 2 |
| IOH 7261 | NM_001639 | 25.4 | 1 | 2 |
| IOH 3413 | NM_001780 | 25.6 | 4 | 3 |
| IOH 10645 | NM_000023 | 42.8 | 2 | 2 |
| IOH 11371 | NM_005755 | 25.4 | 2 | 2 |
| IOH 4919 | NM_013995 | 44.9 | 3 | 10 |

Expression of genes (gp120and the six human ORFs ) was driven by the T7 promoter. An omega 5′ UTR from Tobacco Mosaic Virus was located upstream of the mentioned genes. In vitro transcription was performed using a commercially available transcription kit (i.e., mMessage mMachine T7 Ultra (Ambion)). The resulting m G(5′)pppG(5′)-capped transcripts were purified by LiCl precipitation and a single ethanol wash.

The cell-free translation reactions were performed in a total volume of 25 μL and contained 8 μL of extract (32%). The final concentrations of the other reagents were as follows: 30 mM Hepes/KOH pH 8, 25 μM of each of the 20 amino acids including [³⁵S]Met (0.4 μCi/μL), 1.6 mM Mg(OAc)₂, 100 mM KOAc, 2.5 mM DTT, 0.25 mM spermidine, 1.75 mM ATP, 0.25 mM GTP, 10 mM creatine phosphate, 1 mg/mL creatine kinase, 0.8 units/μL recombinant ribonuclease inhibitor RnaseOut, and 10-20 pmol/μL capped mRNA. Incubation is performed at 25° C. for 2 h. Aliquots of translation product samples were subjected to PNGase F treatment (NEB, Ipswich, Mass.). Translation products were resolved by SDS-PAGE. The gel was exposed to a film overnight.

The results showed that five of these proteins exhibited clear gel shift effects upon cleavage with PNGase F, indicating that these proteins were glycosylated at asparagine residues (FIG. 1). The magnitude of the gel shift correlated well with the number of predicted glycosylation sites. A modified version of gp120 deprived from its signal sequence exhibited no glycosylation (not shown), suggesting that the protein must be first targeted to endogenous microsomes in order to be glycosylated.

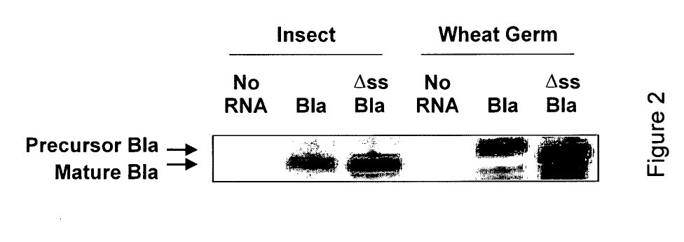

To determine whether the system provided signal sequence cleavage, we used as a reporter E. coli beta-lactamase, a 286 amino acid protein, whose precursor has a 23-amino acid well-characterized signal sequence. This protein and its truncated version deprived from its first 23 amino acids were expressed using both our insect and the Proteios wheat germ translation systems (Invitrotech, Japan). The results showed that while the wheat germ lysate exhibited no signal sequence processing activity, full-processed beta-lactamase was obtained using the insect-based extract (FIG. 2).

The insect cell-free translation extract made by the provided methods resulted in efficient N-linked glycosylation and signal sequence processing.

### Example 3

### Translation System Using BHK Cell Extracts

A 1 ml pellet of freshly harvested cells of the adhesive BHK cell line was resuspended and washed three times with 5 mls of wash buffer (35 mM Hepes-KOH pH 7.5, 146 mM NaCl, 11 mM D-glucose) using 700 rpm centrifugations between washes. The final pellet was raised in two volumes of resuspension buffer (25 mM Hepes pH 7.5, 50 mM KCl, 1.5 mM MgCl₂, 1 mM DTT) and the cell resuspension was pipetted into mini-bomb. The extract was left under 200 psi for 25 min. at 4° C. (˜1.5 ml). The lysed cells were then collected. In alternate procedures, the cells were lysed by 15 strokes in a glass homogenizer. One-tenth volume of 10× post lysis buffer (25 mM Hepes pH7.5, 1M KC₂H₃O₂, 30 mM MgCl₂ and 30 mM DTT) was added and the lysate was centrifuged for 20 minutes at 14,000 rpm in a microcentrifuge. The supernatant was removed and frozen at −80° C.

The constructs used in the study were pFK1009, a construct based on the pEU3NII vector (Proteios, Invitrotech, Japan) that includes the T7 promoter followed by the TMV omega sequence, followed by a multiple cloning site, into which the luciferase gene was cloned, and pSP-luc, from Promega's luciferase assay system, which includes the luciferase gene but lacks the TMV omega sequence. Genes were transcribed using T7 or SP6 polymerase using the cap m⁷G(5′)pppG(5′).

Translations were performed using creatine kinase 5 mg/ml (0.5 ul), Buffer #2 Proteios wheat germ system (1.5 ul), RNaseOut (0.25 ul), Buffer #1 (0.85 ul), 35Smet (0.5 ul), and BHK extract (6 ul). The translation reactions were incubated at 33° C. for 1 hour. 2.5 ul of each translation reaction was used for luciferase analysis.

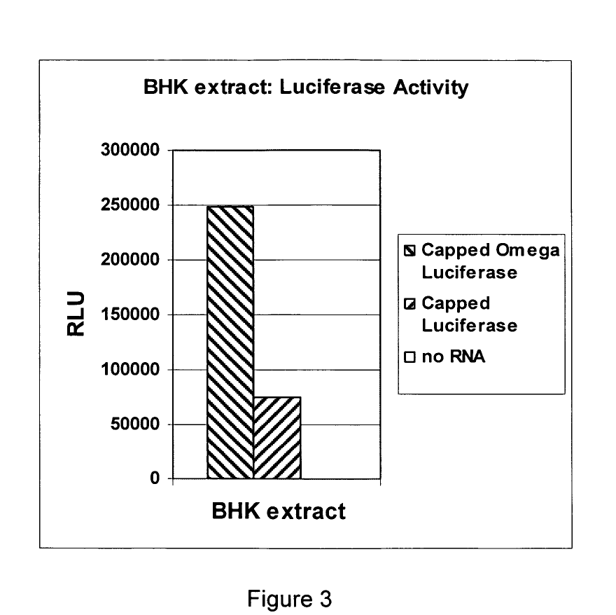

FIG. 3 depicts the translational efficiency of the cell free system using capped RNA transcripts with and without the TMV omega sequence. The TMV omega sequence enhances translation by at least three-fold.

The entirety of each patent, patent application, publication and document referenced herein hereby is incorporated by reference, including all tables, drawings, and figures. All patents and publications are herein incorporated by reference to the same extent as if each was specifically and individually indicated to be incorporated by reference. Citation of the above patents, patent applications, publications and documents is not an admission that any of the foregoing is pertinent prior art, nor does it constitute any admission as to the contents or date of these publications or documents. All patents and publications mentioned herein are indicative of the skill levels of those of ordinary skill in the art to which the invention pertains.

Modifications may be made to the foregoing without departing from the scope, spirit and basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. One skilled in the art readily appreciates that the present invention is well adapted to carry out the objects and obtain the ends and advantages mentioned, as well as those inherent therein. The examples provided herein are representative of specific embodiments, are exemplary, and are not intended as limitations on the scope of the invention.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

1. A composition which comprises a cell-free extract and an isolated ribonucleic acid comprising an exogenous cap, a 5′ untranslated ribonucleotide sequence comprising an translational enhancer ribonucleotide sequence, and a target ribonucleotide sequence, wherein the cell-fee extract is derived from insect cells, avian cells, or mammalian cells.

2-15. (canceled)

16. The composition of claim 1, wherein the cap is selected from the group consisting of caps in Table 1.

17. The composition of claim 1, wherein the cap comprises a methylated nucleotide base.

18. The composition of claim 1, wherein the cap is m⁷C(5′)pppG(5′).

19-60. (canceled)

61. A process for preparing a cell-free extract for in vitro translation from an insect cell, avian cell, or mammalian cell, which consists essentially of rupturing insect cells or mammalian cells and removing intact cells, whereby a cell-free extract is prepared.

62. The process of claim 61, wherein the cell-free extract comprises functional components for glycosylation.

63. (canceled)

64. A process for synthesizing a target protein or target peptide in a cell-free system, comprising:
contacting a nucleic acid that encodes a target protein or target peptide with a cell-free extract,
wherein the cell-free extract is prepared by a process which consists essentially of rupturing insect cells or mammalian cells and removing intact cells,
whereby the target protein or target peptide is synthesized.

65. The process of claim 64, wherein the protein or peptide is glycosylated.

66. (canceled)

67. (canceled)

68. The process of claim 61 wherein the cells are ruptured at a pressure of about 150 pounds per square inch or greater.

69. The process of claim 61, wherein the cells are ruptured at a pressure of 500 pounds per square inch or greater.

70. The process of claim 61, wherein the cells are ruptured at a pressure of 1000 pounds per square inch or greater.

71. The process of claim 61, wherein the cells are ruptured at a pressure of 10000 pounds per square inch or greater.

72. The process of claim 61, wherein the cells are ruptured by mechanical shear.

73. The process of claim 64. wherein the nucleic acid is a deoxyribonucleic acid.

74. The process of claim 73, wherein the deoxyribonucleic acid encodes a ribonucleic acid comprising a 5′ untranslated ribonucleotide sequence comprising a translational enhancer ribonucleotide sequence and a target ribonucleotide sequence.

75. The process of claim 64, wherein the nucleic acid is a ribonucleic acid.

76. The process of claim 75, wherein the ribonucleic acid comprises a 5′ untranslated ribonucleotide sequence comprising an translational enhancer ribonucleotide sequence, and a target ribonucleotide sequence.

78. The process of claim 75, wherein the ribonucleic acid comprises a cap.

79. A process for cell-free translation of a protein or peptide, comprising:
contacting in a system a cell-free extract derived from non-rabbit mammalian cells or insect cells with a ribonucleic acid comprising a 5′ untranslated region comprising an translational enhancer ribonucleotide sequence and a target ribonucleotide sequence;
whereby a protein or peptide is translated from the target ribonucleotide sequence.

80. The process of claim 79, wherein the ribonucleic acid comprises an exogenous cap.

81-108. (canceled)

109. The composition of claim 1, wherein the 5′ untranslated ribonucleotide sequence comprises a tobacco mosaic virus omega sequence, a translational enhancer substantially homologous to a tobacco mosaic virus omega sequence, or a translational enhancer related to a tobacco mosaic virus omega sequence.
